(19) United States
(12) Patent Application Publication
(10) Pub. No.: US 2009/0217400 A1
(43) Pub. Date: Aug. 27, 2009

(54) Enzymes, cells and methods for site specific recombination at asymmetric sites

(75) Inventors: Nir Carmi, Ramat Efal (IL); Yoram Eyal, Mesilat Zion (IL); David Gidoni, Shani-Livna (IL); Peter G. Schultz, La Jolla, CA (US); Stephen W. Santoro, Cambridge, MA (US)

Correspondence Address: BROWDY AND NEIMARK, P.L.L.C.;624 NINTH STREET, NW, SUITE 300, WASHINGTON, DC, 20001-5303, US

(73) Assignees: State of Israel, Ministry of Agriculture, Agricultural Research Organization, Bet Dagan (IL); The Scripps Research Institute, La Jolla, CA (US)

(21) Appl. No.: 10/590,897

(22) PCT Filed: Feb. 24, 2005
(86) PCT No.: PCT/IL05/00230

§ 371 Date: Dec. 18, 2008

Related U.S. Application Data
(60) Provisional application No. 60/547,436, filed on Feb. 26, 2004.

(51) Int. Cl. A01K 67/027; C12N 9/00; C07H 21/00; C12N 5/10; C12N 1/19; A01H 5/00; A61K 31/7088; A61K 9/127; A61K 45/00; A61P 35/00

(52) U.S. Cl. 800/14; 435/183; 536/23.2; 435/325; 435/254.2; 800/295; 424/450; 424/93.21

(57) ABSTRACT

The present invention relates to enzymes, compositions and methods for catalyzing asymmetric recombination of non-palindromic recombination sites in a cell free system, in isolated cells or in living organisms. The enzymes and methods of the invention are suitable for mediating specific recombinations between DNA sequences comprising specific recombination sites without being limited to strict palindromic symmetry within each recombination site.

### FIELD OF THE INVENTION

The present invention relates to enzymes, compositions and methods for catalyzing asymmetric recombination of non-palindromic recombination sites in a cell free system, in isolated cells or in living organisms. The enzymes and methods of the invention are suitable for mediating specific recombinations between DNA sequences comprising specific recombination sites without being limited to strict palindromic symmetry within each recombination site.

### BACKGROUND OF THE INVENTION

Site-specific recombination systems mediate control of a large variety of critical biological functions in nature, through accurate excision, inversion or integration of defined DNA sequences. Site-specific recombination systems function through specific interactions of recombinase enzymes with their corresponding DNA target sequences.

Two of the most characterized recombinases are the Flp protein of yeast and the Cre protein of bacteriophage P1. These recombinases initiate recombination by binding to a specific recognition site, the frt site for Flp protein the lox site for the Cre protein. These two different recognition sites share a strict palindromic organization consisting of two identical 13-bp inverted repeats surrounding a spacer region, 8 bp in length, that confers directionality to the site and hence to the recombination reaction. Each of the palindrome halves provides a binding site for one recombinase monomer, while the recombination reaction takes place within the spacer region. A complete recombinase-mediated recombination takes place between two recombination sites, each bound by two recombinase monomers. The resulting four monomers are brought together by protein-protein interactions forming a homotetrameric assembly of identical recombinase molecules and executing the specific recombination. Symmetric recombinations, particularly insertions, are often reversible in the presence of recombinases.

Albert et al. (The Plant Journal, 7:649-659, 1995) report the use of mutant lox sites, obtained by abbreviating the left and right 13-bp element that flanks the asymmetric spacer, for recombination using wild type Cre. The system of Albert et al. identified three sets of mutant lox sites that enable site-specific symmetric integration of exogenous DNA into the plant genome with a reduced frequency of the reversible process, namely excision the exogenous DNA.

Lee et al. (Gene, 216:55-65, 1998) analyzed the role of each nucleotide sequence of the spacer region in the recombination process by constructing loxP spacer mutants with single-base or double-base substitutions (e.g. SEQ ID NOS: 1-33; Table 2), which may facilitate recombination with diverse efficiencies.

Shaikh et al. (J. Mol. Biol. 302:24-48, 2000) discloses non-palindromic recombination sites comprising sequences derived from lox and frt sites that can be recognized and cleaved by chimeric Cre/Flp recombinases. The chimeric Cre/Flp recombinases can not complete recombination of the non-palindromic sites, apparently due to lack of ligation of the cleavage sites.

A recent publication by some of the inventors of the present invention discloses Cre variants, designated CM1 and CM2 (Table 1), which recognize wild type and/or variant loxP sites (SEQ ID NOS:35-37), and a method for identifying recombinase variants by a directed evolution strategy (Santoro et al., Proc. Natl. Acad. Sci. USA, 99: 4185-4190, 2002).

U.S. Pat. No. 6,465,254 discloses mutant loxP sites and methods of using thereof. However, recombination is performed only between two identical mutant loxP recombination sites.

Methods for recombination in plants using a nucleotide sequence flanked between two non-identical however palindromic recombination sites are disclosed in U.S. Pat. Nos. 6,573,425 and 6,664,108. U.S. Pat. No. 6,573,425 relates to methods of integrating into plants a nucleotide sequence flanked between two non-identical mutant recombination sites thus suppressing excision of said nucleotide sequence, post-integration, in the presence of a recombinase. The differences between the mutant recombination sites and the wild type sites are in the sequence of the spacers. Recombination of the non-identical mutant recombination sites is performed by wild type recombinases. U.S. Pat. No. 6,664,108 relates to Agrobacterium-mediated transfer of T-DNA to a plant cell, wherein the T-DNA contains a viral replicon flanked by recombination sites for a site-specific wild type recombinase, the recombination sites comprising mutant spacer sequences and/or additional restriction sites outside of each palindromic repeat.

Flowers et al. (J. Virol. 1997, 71:2685-2692) demonstrated the potential of Cre-mediated recombination as an antiviral genetic approach to HIV. Flowers et al. showed that wild type Cre provides intracellular inhibition of replication of recombinant HIV-1 containing wild-type loxP by mediating excision of the HIV proviral DNA located between wild-type loxP sites.

There is an unmet need for recombination systems that are not limited to wild type recognition sites and moreover that are not restricted to palindromic symmetry of recognition sites, thus enabling recombination of any desired recombination site.

### SUMMARY OF THE INVENTION

The present invention relates to enzymes capable of mediating site-specific recombination on asymmetric sites and methods for mediating successful recombination events with increased site specificity. The enzymes and methods of the present invention are directed for catalyzing recombination at predetermined genomic loci without being limited to a particular palindromic organization. The present invention further relates to cells obtained by asymmetric recombinations.

The present invention is based in part on the unexpected finding that a composition comprising two distinct recombinase proteins, one of which is a wild type recombinase and the other is a mutant recombinase, is capable of catalyzing recombination of non-palindromic recombination sites with high efficiency. The recombination efficiency and site-specificity of such composition was found to be higher than the recombination efficiency and specificity of a composition consisting exclusively or predominantly the wild type recombinase.

The main drawback of recombination systems known in the art is the requirement for precise palindromic recombination sites that can be identified by the wild type recombinases. The major advantage of the enzymes of the present invention is that they catalyze recombination between recombination sites, wherein at least one recombination site is a non-palindromic recombination site. The enzyme or plurality of enzymes of the present invention may catalyze recombination between DNA molecules comprising recombination sites of any desired sequence with the proviso that the recombination sites can be recognized by said enzyme or plurality of enzymes for the purpose of recombination.

Another major limitation of symmetric recombination is that recombination events are freely reversible. Namely, in integration events where a first DNA molecule integrates into a second DNA molecule, excision of said first DNA readily occurs since both molecules contain similar recombination sites that take part in both excision and insertion. The advantage of the recombinases enzymes of the present invention is that they may catalyze recombination between a first and a second DNA molecule, wherein each DNA molecule comprises different recombination sites, wherein at least one recombination site is an asymmetric recombination site. Thus, the reversed recombination event cannot readily occur as it requires sufficient amount of at least one recombinase, other than wild type recombinases, said at least one recombinase is capable of mediating site-specific recombination on asymmetric sites. Furthermore, the concentration of at least one recombinase can be manipulated such that the desired recombination event is favored over the reversed event.

Without wishing to be bound by any particular theory or mechanism of action, the asymmetric recombination according to the present invention may be attributed to the ability of a plurality of different enzymes to form a heterotetramer thereby bringing the two recombination sites together, facilitating recombination. Alternatively, the asymmetric recombination according to the present invention may be attributed to the ability of a single enzyme to recognize various recombination sites including at least one non-palindromic recombination site, and to form a homotetramer by bringing these recombination sites together, thereby facilitating recombination. This mechanism may be supported by the fact that the DNA binding domain and the catalytic domain within Cre, reside in two distinct and independent locations on the protein.

It is to be understood explicitly that the scope of the present invention encompasses any form of recombination event including, without limitation, recombination between recombination sites that are in a cis or trans location. In the former situation, the orientation of the recombination sites may be the same or the opposite. In the case of trans localization, the DNA strands involved can be linear or circular. In the case of cis location of two recombination sites the outcome of the recombination may be excision or inversion of an intervening sequence. In the case of trans located recombination sites the outcome may be insertion of one DNA into another or translocation between two DNA molecules.

The present invention further provides methods for gene therapy comprising introducing into a subject in need thereof, at least one enzyme or a polynucleotide encoding same, wherein the at least one enzyme is capable of facilitating site-specific recombination on asymmetric sites at a desired genomic locus. Alternatively, the method comprises introducing into a cell said at least one enzyme or a polynucleotide encoding same thereby modifying the cellular genome and further transplanting into an individual in need thereof the genetically modified cell.

The major drawback of gene therapies as known in the art is that the exact genomic location into which a desired gene fragment is introduced is not known. This uncertainty has dangerous and even lethal consequences. The methods of the present invention provide a targeted recombination and are based on a-priori determination of the insertion or excision genomic locus. Following identification of a desired locus, a recombinase or a plurality of recombinases is selected from a library of recombinases, for example the library disclosed in Santoro et al. (ibid), which can catalyze recombination at the desired locus. The reaction may be catalyzed by at least one recombinase or at most four recombinases, wherein each of the four recombinases recognizes one half of a recombination site. Such methods are suitable for treating various diseases including diseases that require excision of an intervening sequence. Thus, the methods may be applied for inhibiting the activity of human immunodeficiency virus (HIV). The DNA-genome of HIV, converted from the RNA-genome of the virus after penetration into the target cell, integrates randomly into the cellular genome as a provirus flanked by long terminal repeats (LTRs). Transcription of the viral genome is initiated at the 5 LTR (the viral promoter) and it is terminated at the 3 LTR, whereas a specific sequence, the R-element, is present at both ends of all viral transcripts. Thus, inhibition of HIV replication by site specific asymmetric excision may be advantageously achieved using the recombinases and methods of the present invention.

In the specification and claims that follow, the terms “site-specific recombination on asymmetric sites” and “asymmetric recombination” are used interchangeably herein to describe recombination between two recombination sites, wherein at least one of the recombination sites is an asymmetric recombination site. In one embodiment, both recombination sites are asymmetric. In another embodiment, both recombination sites are similar and asymmetric. In yet another embodiment, the two recombination sites are distinct from each other. During typical recombination, a specific recombination site within a DNA molecule is being cleaved and a new DNA molecule is ligated into the cleaved site.

The terms “asymmetric recombination site” or “chimeric recombination site” as used herein are interchangeable and used to describe a non-palindromic DNA element comprising a first and a second DNA sequence, also termed hereinafter non-palindromic halves. The two non-palindromic halves flank a spacer region which confers directionality to the recombination site and hence to the recombination reaction. The first and second DNA sequences correspond to two recognition sites. In one embodiment, the two non-palindromic halves are recognized by at least one recombinase. In an alternative embodiment, the two non-palindromic halves are recognized by a plurality of recombinases. In yet another embodiment, at least one non-palindromic half is not similar to a natural recognition site, such as the natural frt or loxP sites.

The term “recombinase” as used herein is to be construed in its most general sense and refers to an enzyme or a plurality of enzymes, active fragments or an active variants thereof, capable of identifying recognition sites within recombination sites and thereby capable of catalyzing recombination events. In fact, a recombinase is an enzyme capable of catalyzing cleavage and ligation at particular sites.

According to a first aspect, the present invention provides an isolated enzyme, the at least one isolated enzyme is capable of mediating site-specific recombination between two predetermined recombination sites, wherein at least one of the recombination sites is an asymmetric recombination site.

According to one embodiment, the asymmetric recombination is selected from a group consisting of: inversion of a first DNA molecule encompassed within a second DNA molecule, excision of a first DNA molecule from a second DNA molecule, insertion of a first DNA molecule into a second DNA molecule and translocation between a first DNA molecule and a second DNA molecule.

According to another embodiment, the second DNA molecule is selected from the group consisting of: genomic DNA and circular DNA. According to yet another embodiment, the second DNA molecule is genomic DNA and the first DNA molecule is integrated into predetermined genomic sites selected from the group consisting of: 3′ UTRs, 5′ UTRs, polyA sites and gene promoters.

According to yet another embodiment, the isolated enzyme is a Cre mutant mediating recombination between two recombination sites, wherein at least one recombination site is an asymmetric recombination site comprising a spacer sequence selected from the group consisting of: SEQ ID NOS. 1-34.

It is to be understood that the enzymes of the present invention are other than CM1 and CM2 enzymes. These two enzyme are disclosed herein solely for the purpose of exemplifying the principles of the present invention.

According to another aspect, the present invention provides a plurality of isolated enzymes, wherein the plurality of isolated enzymes is capable of mediating site-specific recombination between two predetermined recombination sites, wherein at least one of the recombination sites is an asymmetric recombination site. According to one embodiment, each of the plurality of enzymes recognizes at least one half of the at least one asymmetric recombination site.

According to yet another aspect, the present invention provides an isolated polynucleotide encoding at least one enzyme, the at least one enzyme is capable of mediating site-specific recombination between two recombination sites, wherein at least one of the recombination sites is an asymmetric recombination site. According to one embodiment, the isolated polynucleotide is encompassed in a recombinant vector that expresses the at least one recombinase. According to another embodiment, the recombinant vector is selected from the group consisting of: naked DNA plasmid, a plasmid within a liposome, a retroviral vector, an AAV vector, or a recombinant adenoviral vector. According to yet another embodiment, the recombinant vector further comprising a promoter, the promoter is derived from bacteria, yeast, insect, animal, plant or virus. The promoter may be selected from the group consisting of: E. coli lac and trp operons, the tac promoter, the bacteriophage_{λ}L promoter, bacteriophage T7 and SP6 promoters, β-actin promoter, insulin promoter, human cytomegalovirus (CMV) promoter, HIV-LTR, RSV-LTR, SV40 promoter, baculoviral polyhedrin and p10 promoter.

According to yet another embodiment, the promoter is an inducible promoter. The inducible promoter may be selected from the group consisting of: tetracycline, heat shock, steroid hormone, heavy metal, phorbol ester, adenovirus E1A element, interferon, and serum inducible promoters. According to yet another embodiment, the recombinant vector further comprises a nuclear localization signal (NLS) operably linked to the polynucleotide sequence encoding the at least one enzyme. According to one embodiment, the enzyme is a Cre mutant mediating recombination between two recombination sites, wherein at least one recombination site is an asymmetric recombination site comprising a spacer sequence selected from the group consisting of: SEQ ID NOS. 1-34.

According to some embodiments, the isolated polynucleotide encodes a plurality of enzymes, wherein the plurality of isolated enzymes is capable of mediating site-specific recombination between two predetermined recombination sites, wherein at least one of the recombination sites is an asymmetric recombination site. According to other embodiments, each of the plurality of enzymes recognizes at least one half of the at least one asymmetric recombination site.

According to yet another aspect, the present invention provides a host cell comprising a vector encompassing the polynucleotide sequences of the invention for the purposes of storage, propagation, enzyme production and therapeutic applications.

According to yet another aspect, the present invention provides a genetically modified cell transformed by site-specific recombination on at least one asymmetric site, wherein the asymmetric recombination is selected from the group consisting of: inversion, excision, insertion and translocation.

According to one embodiment, the recombination event occurs between the cellular endogenous genome and an exogenous DNA molecule.

According to one embodiment, the genetically modified cell is obtained by integration, such that said genetically modified cell comprises an exogenous DNA molecule, wherein the DNA molecule is integrated by recombination into a predetermined recombination site within the genome of the cell.

According to some embodiments, the genetically modified cell is eukaryotic. According to other embodiments, the genetically modified cell is selected from the group consisting of: yeast, plant cell, mammalian cell, embryonic stem cell, mesenchymal cell, and haematopoietic progenitor cell.

According to certain embodiments, the present invention provides a transgenic organism comprising said genetically modified cell. According to one embodiment, the organism is selected from the group consisting of: plant, yeast, or a vertebrate.

According to an alternative embodiment, the genetically modified cell is obtained by excision, such that the cell is devoid of an endogenous polynucleotide sequence at a predetermined genomic locus.

According to yet another aspect, the present invention provides a method for treating a disease, comprising:
a. providing a composition comprising a DNA molecule comprising a nucleotide sequence encoding at least one recombinase, the at least one recombinase mediates site-specific excision of a gene fragment flanked between two recombination sites, wherein at least one recombination site is an asymmetric recombination site; and
b. administering the composition to a subject in need thereof.

According to one embodiment, the method further comprising obtaining site-specific excision of the gene fragment at a predetermined genomic locus.

According to some embodiments, the composition further comprises a carrier operably connected to the isolated DNA molecule, the carrier capable of targeting said isolated DNA molecule to a cell. According to certain embodiments, the carrier promotes internalization of said isolated DNA molecule into the cell.

According to other embodiments, the carrier is selected from the group consisting of: viruses, liposomes, lipid/DNA complexes, micelles, protein/lipid complexes, nanoparticles, and microparticles.

According to yet another embodiment, the two recombination sites are the same asymmetric recombination sites.

According to yet another embodiment, the nucleotide sequence encodes a plurality of recombinases capable of catalyzing the asymmetric recombination.

According to yet another embodiment, the disease is HIV infection. According to certain embodiments, the excised gene fragment is a fragment of HIV genomic DNA. According to yet another embodiment, the method provides inhibition of HIV replication or elimination of HIV.

According to an alternative embodiment, the composition comprises a recombinant vector encompassing an expression cassette comprising the nucleotide sequence. According to another embodiment, the vector is selected from the group consisting of: naked DNA plasmid, a plasmid within a liposome, retrovirus, lentivirus, adenovirus, herpes simplex viruses (HSV), cytomegalovirus (CMV), and adeno-associated virus (AAV).

According to an alternative embodiment the method comprising:
a. providing a composition comprising an isolated DNA molecule comprising a nucleotide sequence encoding at least one recombinase, the at least one recombinase mediates excision of a gene fragment having two recombination sites, wherein at least one of the recombination sites is an asymmetric recombination site; and
b. transforming a cell with the composition.

According to one embodiment the method further comprising proliferating the transformed cells ex vivo. According to another embodiment, the cell is autologous. According to yet another embodiment the method further comprising obtaining site-specific excision of the gene fragment at a defined genomic locus within the cell. According to yet another embodiment, the method further comprising selecting cells devoid of said gene fragment. According to yet another embodiment the method further comprising transplanting the selected cell into a subject in need thereof.

According to yet another embodiment, transforming the cell with said composition is carried out by a procedure selected from the group consisting of: calcium phosphate transfection, DEAE-dextran mediated transfection, transvection, microinjection, cationic lipid-mediated transfection, electroporation, scrape loading, ballistic introduction or infection, use of a gene gun, and lyposome transfection.

According to another alternative embodiment, the composition comprising at least one recombinase, the at least one recombinase mediates excision of a gene fragment having two recombination sites, wherein at least one of the recombination sites is an asymmetric recombination site. According to yet another embodiment, the composition further comprising a carrier operably linked to said at least one recombinase, the carrier is capable of targeting said at least one recombinase to a specific cell and promoting internalization of said at least one recombinase into the specific cell.

According to yet another embodiment, the method comprising:
a. providing a composition comprising: a first DNA molecule comprising a first recombination site; and a second DNA molecule comprising a nucleotide sequence encoding at least one recombinase, the at least one recombinase mediates insertion of the nucleotide sequence into a third DNA molecule comprising a second recombination site; and
b. administering the composition to a subject in need thereof.

According to one embodiment, the first DNA molecule comprises a nucleotide sequence consisting of a fragment of human genomic DNA. According to another embodiment, the first DNA molecule is a gene for: a structural protein, an enzyme, or a regulatory molecule. According to yet another embodiment, the third DNA molecule is genomic DNA. According to yet another embodiment, the first DNA molecule is inserted into a defined locus of the genome selected from the group consisting of: 3′ UTRs, 5′ UTRs, polyA sites and gene promoters.

According to some embodiments, the composition further comprises a carrier operably connected to the first and second DNA molecules, the carrier capable of targeting said first and second DNA molecules to a cell. According to some embodiments, the carrier is selected from the group consisting of: viruses, liposomes, lipid/DNA complexes, micelles, protein/lipid complexes, nanoparticles, and microparticles.

According to another embodiment, the first DNA molecule and the second DNA molecule are operably linked to one another. According to yet another embodiment, the second DNA molecule is operably linked to a promoter.

According to yet another alternative embodiment the method comprising: transforming a cell with the composition. According to one embodiment the method further comprising proliferating the transformed cells ex vivo. According to another embodiment, the cell is autologous. According to yet another embodiment the method further comprising obtaining site-specific excision of the gene fragment at a defined genomic locus within the cell. According to yet another embodiment, the method further comprising selecting cells devoid of said gene fragment. According to yet another embodiment the method further comprising transplanting the selected cell into a subject in need thereof.

According to one embodiment, the two recombination sites are the same asymmetric recombination sites. According to another embodiment, the second DNA molecule comprises a nucleic acid encoding a plurality of recombinases capable of catalyzing the asymmetric recombination. According to yet another embodiment, at least one recombination site comprises a spacer consisting of any one of the sequences set forth in SEQ ID NOS: 1-34.

According to yet another embodiment, the first DNA molecule comprises a recombination site comprising SEQ ID NO:37 and the second DNA molecule comprising a nucleotide sequence encoding CM2 Cre mutant.

Other objects, features and advantages of the present invention will become clear from the following description and drawings.

### BRIEF DESCRIPTION OF THE DRAWINGS

FIG. 1 demonstrates SEQ ID NOS.:35-38 (A), SEQ ID NOS.:39-44 (C) and oligos (B) used for creating left flanking and right flanking loxP-M7 sites on either side of a ˜1-kb fragment that was utilized to prepare the loxP-M7 substrate (see FIG. 2).

FIG. 2 presents a membrane (A) and a quantitative representation thereof (B) of in vitro recombination obtained by incubating 4 kb linear fragment containing LoxP-M7 (SEQ ID NO:37) and various recombinase: a. wt Cre, b. CM2, c. a mixture of 50% CM2 and 50% wt Cre, and d. CM1.

FIG. 3 shows in vitro recombination activity as a function of reaction time, obtained by incubating linearized Bluescript™ loxP-M7 plasmid (4 kb) and various recombinase at three different total recombinase concentrations: A, 30 nM; B, 60 nM and C, 90 nM.

FIG. 4 represents a schematic model of recombination by a heterotetrameric Cre assembly. Black- and gray-shaded ellipses represent wt Cre and CM2 monomers, respectively, each bound to a lox half-site. Black-shaded strands represent the loxP half-site of loxP-M7. Gray-shaded strands represent the lox M7 half-site of loxP-M7.

FIG. 5 exhibits lox-LTR sequences.

FIG. 6 shows schematic representations (A-C) of a strategy for selecting Cre mutants capable of catalyzing asymmetric recombinations.

### DETAILED DESCRIPTION OF THE INVENTION

### Definitions

The terms “sequence-specific recombinase” and “site-specific recombinase” refer to enzymes that recognize and bind to a specific recombination site or sequence and catalyze the recombination of nucleic acid in relation to these sites.

The terms “sequence-specific recombinase target site” and “site-specific recombinase target site” refer to short nucleic acid site or sequence which is recognized by a sequence- or site-specific recombinase and which become the crossover regions during the site-specific recombination event. Examples of sequence-specific recombinase target sites include, but are not limited to, lox sites, frt sites, ATT sites and DIF sites. According to a currently preferred embodiment, the target sites are asymmetric recombination sites, wherein each asymmetric recombination site comprises a first and second non-palindromic halves flanking a spacer region which confers directionality to the recombination site and hence to the recombination reaction.

The term “spacer” is to be construed in its most general sense and refers to an asymmetric core sequence consisting of 8 bp sequence located between the two half of a recombination site.

The term “lox site” as used herein refers to a nucleotide sequence at which the product of the cre gene of bacteriophage P1, Cre recombinase or mutants thereof, can catalyze a site-specific recombination. This term further encompasses a variety of lox sites are known to the art including the naturally occurring loxP (the sequence found in the P1 genome), loxB, loxL and loxR (these are found in the E. coli chromosome) as well as a number of mutant or variant lox sites such as loxP511, loxΔ86, loxΔ117, loxC2, loxP2, loxP3 and loxP23. The loxP site comprises two 13 bp inverted repeat sequences separated by an 8 bp spacer region (Hoess et al., Proc. Natl. Acad. Sci. USA 79:3398, 1982). The internal spacer sequence of the loxP site is asymmetrical and thus, two loxP sites can exhibit directionality relative to one another (Hoess et al. Proc. Natl. Acad. Sci. USA 81:1026, 1984). When two loxP sites on the same DNA molecule are in a directly repeated orientation, Cre excises the DNA between these two sites leaving a single loxP site on the DNA molecule. (Abremski et al. Cell 32:1301, 1983). If two loxP sites are in opposite orientation on a single DNA molecule, Cre inverts the DNA sequence between these two sites rather than removing the sequence. The Cre recombinase also recognizes a number of variant or mutant lox sites relative to the loxP sequence. Examples of these Cre recombination sites include, but are not limited to, the loxB, loxL and loxR sites which are found in the E. coli chromosome.

The term “frt site” as used herein refers to a nucleotide sequence at which the product of the FLP gene of the yeast 2 micron plasmid, FLP recombinase, can catalyze a site-specific recombination.

As used herein, the term “vector” is used in reference to nucleic acid molecules that transfer DNA segment(s) from one cell to another. The term “vehicle” is sometimes used interchangeably with “vector.” A “vector” is a type of “nucleic acid construct.” The term “nucleic acid construct” includes circular nucleic acid constructs such as plasmid constructs, plasmid constructs, cosmid vectors, etc. as well as linear nucleic acid constructs (e.g., λ-phage constructs, PCR products). The nucleic acid construct may comprise expression signals such as a promoter and/or an enhancer (in such a case it is referred to as an expression vector).

The term “expression vector” as used herein refers to a recombinant DNA molecule containing a desired coding sequence and appropriate nucleic acid sequences necessary for the expression of the “operably linked” coding sequence in a particular host. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The term “transforming” refers to DNA transfer to a host, achieved by any method known in the art, including but not limited to, transfection of DNA by calcium phosphate-precipitates, conventional mechanical procedures such as microinjection, electroporation or lipofection, insertion of a plasmid encapsulated in liposomes and use of virus vectors.

The term “host cell” refers to cells capable of growth in culture and capable of expressing an enzyme or a plurality of enzymes capable of mediating site-specific recombination between two predetermined recombination sites, wherein at least one of the recombination sites is an asymmetric recombination site. The host cells of the present invention includes prokaryotic, eukaryotic, and insect cells. A host cell strain may be chosen which modulates the expression of the inserted sequences, or modifies and processes the gene product in the specific fashion desired. Expression from certain promoters can be elevated in the presence of certain inducers (e.g., zinc and cadmium ions for metallothionine promoters). Therefore expression of the enzyme or plurality of enzymes of the invention may be controlled. Different host cells have characteristic and specific mechanisms for the post-translational processing and modification of protein. Appropriate cell lines or host systems can be chosen to ensure the correct processing of enzymes expressed.

The term “administering” as used herein incorporates the common usage and refers to any appropriate means to give a pharmaceutical to a patient, taking into consideration the pharmaceutical composition and the preferred site of administration. Preferably, the term “administering” encompasses targeting the composition of the invention to a desired population of cells, wherein the cells are capable of internalizing said composition and thereby to express or host the at least one recombinase of the invention.

The scope of the present invention encompasses asymmetric recombination between recombination sites that are in a cis or trans location. As used here, the term “cis” refers to genetic changes that are on the same DNA molecule in simple organisms or in the same haploid genome in cases where there are multiple chromosomes. The term “trans” refers to genetic changes that are carried by different genomes that have been introduced into the same cell (in any of the possible ways discussed above).

### PREFERRED MODES FOR CARRYING OUT THE INVENTION

The ability to direct site-specific recombination into natural sites in the eukaryotic genome, using the existing means and methodologies, is limited particularly due to the strict prerequisite for palindromic recombination sites. The present invention provides compositions and methods that provide a versatile solution to such limitation as recombination according to the present invention is enabled with the involvement of non-palindromic recombination sites.

Thus, the teaching of the present invention broadens the prospects for genetic manipulation of the eukaryotic genome, enabling integration, deletion or replacement of specific genes and DNA segments in defined genomic loci.

It is to be understood that according to the teaching of the present invention, the site for insertion is preferably selected prior to insertion and is used for screening for recombinase(s) that recognize said site at adequate specificity. The site for insertion may be identified in silico and must comprise a core spacer sequence. For example, screening for a Cre mutant that can catalyze the asymmetric recombination is performed using, an asymmetric recombination site comprising a spacer sequence having 70% homology, preferably 80% homology to a sequence selected from the group consisting of: SEQ ID NOS: 1-34. Screening for a Cre-mutant may also include an initial selection of recombinases recognizing a new spacer and then a selection of recombinase(s) that recognize the flanking halves.

In a certain embodiment, the present invention provides a composition comprising a Cre variant and a composition comprising a wild type Cre and a Cre variant CM2, which catalyze the recombination of non-palindromic sites. These compositions were shown to catalyze asymmetric recombination in sites which contain mutations within the restricted intolerant non-flexible sequence, i.e. in positions 2-7 f the recombination site, thus demonstrating that not only that the compositions and method of the invention are not restricted to the palindromic symmetry of recombination sites but they are also not to restricted to asymmetric recombination sites having mutations only within their flexible regions.

Thus, according to some embodiments, the composition of the invention comprises a plurality of recombinases which may encompass at least one distinct wild type recombinase. The wild type recombinase may be derived from prokaryotic and eukaryotic sources.

It was previously shown that the Cre protein sequence for DNA recognition of the inverted repeats lox site is independent of the protein sequence which is responsible of the cleavage-ligation on the spacer lox site. Therefore, without wishing to be bound by any particular theory or mechanism of action, asymmetric recombination according to the present invention may be attributed to the fact that mutations in the recombinase sequence, including but not limited to the docking recognition sites, namely the two halves that flank the spacer on the lox site, do not disrupt the cleavage-ligation mechanism. This attribution is supported by Shaikh et al. (ibid) who showed that Cre/FLP chimeras successfully cleaved a chimeric substrate. This mechanism is further supported by the fact that the DNA binding domain and the catalytic domain within Cre, reside in two distinct and independent locations on the protein (Gopaul et al., EMBO J. 1998 17: 4175-4187). The mode of cleavage of Cre was in cis whereas that of FLP was in trans. However, recombination of the chimeric protein did not occur.

### Selection of Recombinases for Asymmetric Recombination

Site directed recombination with at least one asymmetric recombination site, particularly endogenous asymmetric recombination sites, requires selection of recombinases that recognize the asymmetric recombination sites, and further capable of catalyzing the asymmetric recombination.

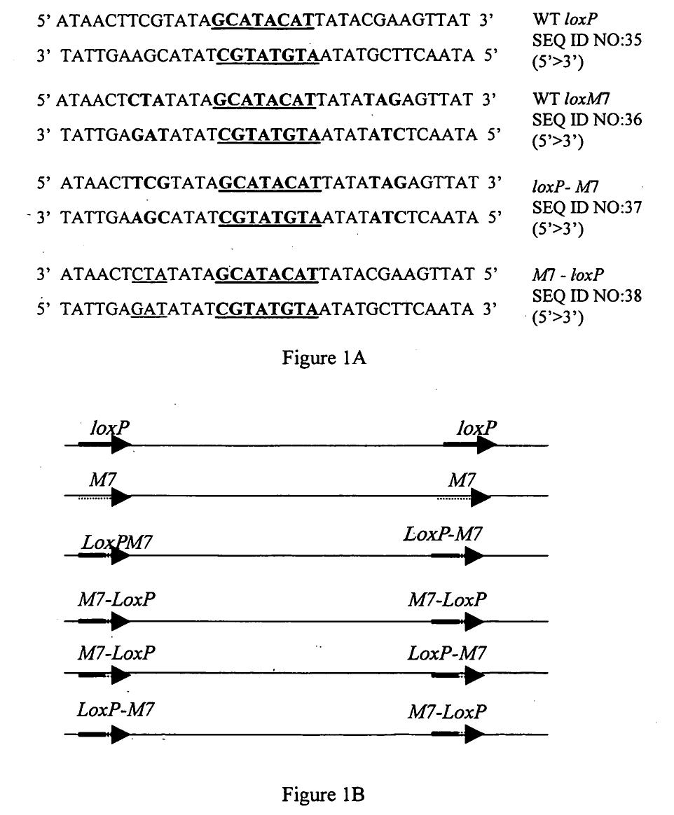

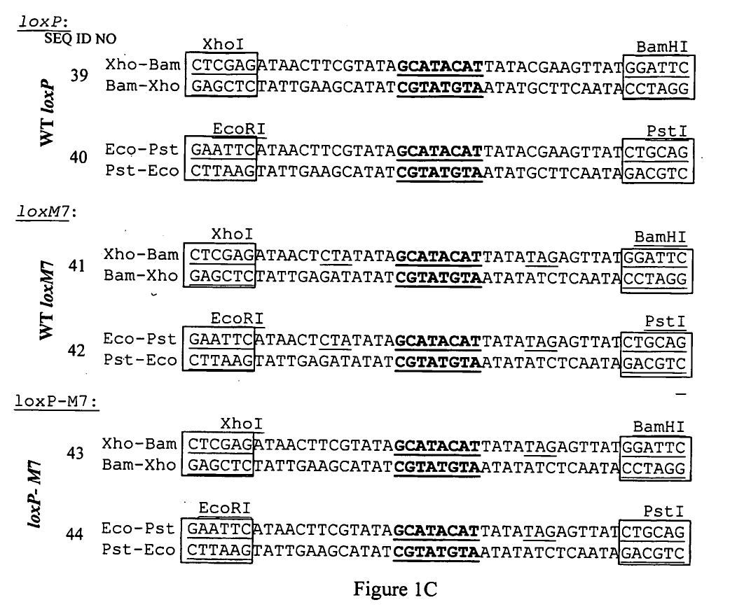

For the selection of new Cre mutants, constructs (such as described in FIG. 1C) are transformed into a library of cells comprising and expressing Cre mutants such as the library disclosed in Santoro et al. (ibid). A new library may be formed for this purpose, by combining two Cre mutants' libraries (Santoro et al., ibid) and shuffling their elements using DNA shuffling methods known in the art (e.g. U.S. Pat. Nos. 6,326,204; 6,479,652 and 6,489,145).

The constructs may comprise fragments selected from a variety of recombination sites. The construct may comprise a spacer selected from the spacers set forth in Table 1 or a novel spacer. In the later event, selection of a recombinase or a plurality of recombinase capable of mediating the desired recombination event may thus include a first step for selecting recombinases recognizing the new spacer and a second step for selecting recombinases recognizing the recognition sites flanking the novel spacer. (see for example, Buchholz, F. and Stewart, A. F., Nat. Biotechnol. 2001, 19:1047-1052)

One half of the asymmetric recombination site of the invention may also be selected from a variety of other recombination sites recognized by recombinases other than Cre. Examples of the non-Cre recombinases include, but are not limited to, site-specific recombinases include: the Int recombinase of bacteriophage, the FLP recombinase of the 2pi plasmid of Saccharomyces cerevisiae, the resolvase family, transposase of Bacillus thruingiensis.

The Int recombinase of bacteriophage λ belongs to the integrase family and mediates the integration of the λ genome into the E. coli chromosome. The Int recombinase of bacteriophage λ promotes irreversible recombination between its substrate ATT sites as part of the formation or induction of a lysogenic state (Landy, A., Ann. Rev. Biochem. 58:913, 1989). Reversibility of the recombination reactions results from two independent pathways for integrative and excessive recombination. Each pathway uses a unique but overlapping set of the 15 protein binding sites that comprise ATT site DNAs. Cooperative and competitive interactions involving four proteins (Int, Xis, IHF and FIS) determine the direction of recombination. Integrative recombination involves the Int and IHF proteins and sites ATT-P (240 bp) and ATT-B (25 bp). Recombination results in the formation of two new sites: ATT-L and ATT-R. Excessive recombination requires Int, IHF, and Xis, and sites ATT-L and ATT-R to generate ATT-P and ATT-B. Derivatives of the ATT site with changes within the 15 bp core may also be suitable for efficient recombination. Integrase can be obtained as described by Nash, H. A., (1983) Methods of Enzymology 100:210-216.

The other members of the Integrase family of site-specific recombinases may also be used to construct libraries of alternative recombination proteins and recombination sites for the present invention. Examples of such Int recombinases include, but not limited to, site-specific recombinase encoded by bacteriophage λ, P22, P2, 186 and P4. This group of recombinases exhibits a large diversity of sequences, but all of the recombinases can be aligned in their C-terminal halves. Three positions are perfectly conserved within this family: histidine, arginine and tyrosine are found at respective alignment positions 396, 399 and 433 within the well-conserved C-terminal region. These residues contribute to the active site of this family of recombinases, and suggest that tyrosine-433 forms a transient covalent linkage to DNA during strand cleavage and rejoining.

The FLP recombinase of the 2pi plasmid of Saccharomyces cerevisiae recognizes the frt site which, like the loxP site, comprises two 13 bp inverted repeats separated by an 8 bp spacer. The FLP gene has been cloned and expressed in E. coli and in mammalian cells and has been purified (e.g. Meyer-Lean et al. Nucleic Acids Res. 15:6469, 1987).

The resolvase family members, such as the Tn3 resolvase, the Hin recombinase, and the Cin recombinase, may also be used for recombination according to the present invention. Transposase of Bacillus thuringiensis may also be used as recombination proteins and recombination sites. Bacillus thuringiensis is an entomopathogenic bacterium whose toxicity is due to the presence in the sporangia of A-endotoxin crystals active against agricultural pests and vectors of human and animal diseases. Most of the genes coding for these toxin proteins are plasmid-bome and are generally structurally associated with insertion sequences.

Other recombination systems may also be used as recombination proteins and recombination sites, including the xerC and xerD recombinases of E. coli which together form a recombinase (e.g. Leslie et al., EMBO J. 14:1561, 1995).

The constructs are preferably encompassed within an expression vector. The recombinant expression vector may optionally include an affinity tag for selection and isolation of protein product encoded by same. Examples of such an affinity tag include, but are not limited to, a polyhistidine tract, polyarginine, glutathione-S-transferase (GST), maltose binding protein (MBP), a portion of staphylococcal protein A (SPA), and various immunoaffinity tags (e.g. protein A) and epitope tags such as those recognized by the EE (Glu-Glu) antipeptide antibodies. The affinity tag may also be a signal peptide either native or heterologous to baculovirus, such as honeybee mellitin signal peptide. The affinity tag may be positioned at either the amino- or carboxy-terminus of the donor DNA.

The constructs may further comprise a promoter sequence that controls the expression of the recombinase. The promoter may be any array of DNA sequences that interact specifically with cellular transcription factors to regulate transcription of the downstream gene. The promoter may be derived from any organism, such as bacteria, yeast, insect and mammalian cells and viruses. The selection of a particular promoter depends on what cell type is to be used to express the protein of interest.

The constructs may further comprises a nuclear localization signal sequence (NLS) which is operably linked to the polynucleotide encoding the at least one recombinase. Compositions comprising the nucleic acid to be transfected and a targeting element which makes it possible to orient the transfer of the nucleic acid, such as a ligand of the intracellular type such as a nuclear localization signal sequence (NLS) are known in the art and may be utilized to direct the polynucleotide of the invention into the nucleus. For example, International Patent Applications WO 94/23751, WO 95/31557, WO 96/25508, WO 97/18317, WO 97/30170, European Patent Application 0,544,292, and U.S. Pat. No. 6,750,058, among others.

Other then containing the necessary elements for the transcription and translation of the inserted coding sequence, the expression constructs or vectors of the present invention can also include sequences engineered to enhance stability, production, purification, yield or toxicity of the expressed recombinases. For example, the expression of a fusion protein comprising the recombinase and a heterologous protein can be engineered. With such design the recombinase can be readily isolated by affinity chromatography; e.g., by immobilization on a column specific for the heterologous protein. Where a cleavage site is engineered between the recombinase moiety and the heterologous protein, the recombinase can be released from the chromatographic column by treatment with an appropriate enzyme or agent that disrupts the cleavage site (e.g., see Booth et al. (1988) Immunol. Lett. 19: 65-70; and Gardella et al., (1990) J. Biol. Chem. 265: 15854-15859).

A variety of prokaryotic or eukaryotic cells can be used as host-expression systems to express the recombinase coding sequence. These include, but are not limited to, microorganisms, such as bacteria transformed with a recombinant bacteriophage DNA, plasmid DNA or cosmid DNA expression vector containing the recombinase coding sequence; yeast transformed with recombinant yeast expression vectors containing the recombinase coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the recombinase coding sequence. Mammalian expression systems can also be used to express recombinase. Bacterial systems are preferably used to produce recombinant recombinase, according to the present invention, thereby enabling a high production volume at low cost.

In bacterial systems, a number of expression vectors can be advantageously selected depending upon the use intended for the recombinase expressed. For example, when large quantities of recombinase are desired, vectors that direct the expression of high levels of protein product, possibly as a fusion with a hydrophobic signal sequence, which directs the expressed product into the periplasm of the bacteria or the culture medium where the protein product is readily purified may be desired.

As discussed hereinabove, certain fusion protein engineered with a specific cleavage site to aid in recovery of the recombinase may also be desirable. Such vectors adaptable to such manipulation include, but are not limited to, the pET series of E. coli expression vectors (Studier et al. (1990) Methods in Enzymol. 185: 60-89).

It will be appreciated that when codon usage for recombinase gene cloned from C. melo is inappropriate for expression in E. coli, the host cells can be co-transformed with vectors that encode species of tRNA that are rare in E. coli but are frequently used by plants. For example, co-transfection of the gene dnaY, encoding tRNA. ArgAGA/AGG, a rare species of tRNA in E. coli, can lead to high-level expression of heterologous genes in E. coli. (Brinkmann et al., Gene 85:109, 1989 and Kane, Curr. Opin. Biotechnol. 6:494, 1995). The dnaY gene can also be incorporated in the expression construct such as for example in the case of the pUBS vector (U.S. Pat. No. 6,270,988).

In yeast, a number of vectors containing constitutive or inducible promoters can be used, as disclosed in U.S. Pat. No. 5,932,447.

Alternatively, vectors can be used which promote integration of foreign DNA sequences into the yeast chromosome.

In cases where plant expression vectors are used, the expression of the recombinase coding sequence can be driven by a number of promoters. For example, viral promoters such as the 35S RNA and 19S RNA promoters of CAMV (Brisson et al., 1984, Nature 310: 511-514), or the coat protein promoter to TMV (Takamatsu et al., 1987, EMBO J. 6:307-311) can be used. Alternatively, plant promoters such as the small subunit of RUBISCO (Coruzzi et al., 1984, EMBO J. 3:1671-1680 and Brogli et al., 1984, Science 224: 838-843) or heat shock promoters, e.g., soybean hspl7.5-E or hspl7.3-B (Gurley et al., 1986, Mol. Cell. Biol. 6:559-565) can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems, which are well known in the art can also be used by the present invention.

In any case, recombinase transformed cells are cultured under effective conditions, which allow for the expression of high amounts of recombinase, or at least the amount required for catalyzing the asymmetric recombination. Effective culture conditions include, but are not limited to, effective media, bioreactor, temperature, pH and oxygen conditions that permit protein production. An effective medium refers to any medium in which a cell is cultured to produce the recombinase of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the fermentation medium; be secreted into a space between two cellular membranes, such as the periplasmic space in E. coli; or be retained on the outer surface of a cell or viral membrane.

Following a certain time in culture, recovery of the recombinant enzyme is effected. The phrase “recovering the recombinant enzyme” refers to collecting the whole fermentation medium containing the protein and need not imply additional steps of separation or purification. Recombinases of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Recombinases of the present invention are preferably retrieved in “substantially pure” form. As used herein, “substantially pure” refers to a purity that allows for the effective use of the protein in the diverse applications, described hereinabove.

Expression determination of the hereinabove described recombinant proteins can be effected using specific antibodies, which recognize the recombinases of the present invention. Aside from their important usage in detection of expression of a recombinase, these antibodies can be used as to screen expression libraries and/or to recover desired enzymes of the present invention from a mixture of proteins and other contaminants.

For screening libraries to select for the expressed recombinase of the present invention, a variety of methods known to those of skill in the art may be used, including directed evolution using selectable antibiotic resistant gene, killer gene or visual selectable marker. The next step is using negative-positive selection for improving specificity.

Once a clone is identified in a screen such as the one described above, it can be isolated or plaque purified and sequenced. The insert may then be used in other cloning reactions, for example, cloning into an expression vector that enables efficient production of the recombinase, as detailed above.

### Application of Asymmetric Recombination

The present invention provides a method for catalyzing asymmetric recombination between recombination sites, wherein at least one recombination site is an asymmetric recombination site. In certain embodiments the method comprising providing a population of cells, genetically modifying the cells by inserting into said cells, or excising from said cells, a DNA sequence in a predetermined locus. The method further comprises selecting a subpopulation of the genetically modified cells.

Following asymmetric recombination according to the present invention, it may be required to select cells that were genetically modified for further applications, including transplanting such cells in a plant or a mammal and regenerating transgenic plants. Selecting the desired subpopulation of genetically modified cells may be achieved by using a selectable marker gene carried by the recombinant expression vector or attached to the exogenous DNA which is integrated into the genomic locus of the genetically modified cells. The expression vector may contain more than one selectable marker to facilitate subsequent identification and selection of clones of cells comprising the recombination product under suitable conditions. The selectable marker may encode any functional element, such as protein, peptide, RNA, binding site for RNA and proteins, or products that provide resistance to organic or inorganic agents. Examples of selectable markers include, but are not limited to, reporter genes such as β-galactosidase (GAL), fluorescent proteins (e.g., GFP, GFP-UV, EFFP, BFP, EBFP, ECFP, EYFP), secreted form of human placental alkaline phosphatase such as SEAP, β-glucuronidase (GUS); resistance genes that encodes products which provide resistance against other wise toxic agents such as antibiotics (e.g. neomycin G418, hygromycin resistant gene and puromycin resistant gene), yeast selectable markers leu2-d and URA3, apoptosis resistant genes (e.g. the baculoviral p35 gene) that encode proteins that binds to products which are detrimental to cell survival and promote apoptosis; antisense-oligonucleotides, and any other DNA that encodes product that directly or directly confer sensitivity of cells to particular agents.

Additional strategy for selection of the desired subpopulation of cells includes applying the cis-trans test. This test provides an assessment as to whether two mutations that occur in different genomes have altered the same unit of function (usually the coding sequence for a single protein). It is expected that when the two mutations are put together in the same genome (cis) that they will not support normal function, even if they occur in two separate functional units. Such a test is straightforward in haploid organisms. However, to see cis lack of function in a diploid organism, it is necessary to make the organism homozygous for the cis genome. Otherwise there will be a wild type genome opposite it that will support normal function. In most cases, the cis part of the cis-trans is an implied control that could be run, rather than an important part of the actual experimental study.

In certain embodiments, selection of genetically modified cells involves not only selection of cells that underwent a successful asymmetric recombination but also cells which are devoid of the recombinase(s) capable of performing said asymmetric recombination. The purpose of the later selection is to obtain a stable subpopulation of cells rather than cell that may undergo the reversed event of recombination due to the presence of said recombinase or said plurality of recombinases.

In some embodiments, the DNA encoding the recombinases of the invention together with a donor DNA that is integrated into the genome as a result of a successful recombination, are administered to a cell or a subject. Both DNA molecules may be encompassed within an expression vector. The recombinant expression vector or the recombinant donor DNA may optionally include an affinity tag for selection as detailed above. Preferably, for insertion the recombinase is expressed transiently.

In the event that the method of the invention is for inserting, by asymmetric recombination, recombinant donor DNA into a predetermined genomic location, the recombinant DNA sequence may be any deoxyribonucleotide sequence encoding a functional gene or any synthetically generated DNA sequence. For example the recombinant DNA segment may be a sequence derived from cDNA of a particular gene or one of the members of a cDNA library. The cDNA library may be produced by converting mRNAs in a sample into double-stranded complementary DNA (cDNA) by using reverse transcriptase (RT) and the Klenow fragment of nucleic acid polymerase I. Depending on the source of mRNA sample, the cDNA library may contain various populations of genes of interest, such as disease genes located in certain tissue or type of cells. The recombinant DNA may also be a genomic DNA that contains the coding region interrupted with non-coding sequences (introns/intervening sequences). These introns may contain regulatory elements such as enhancers.

The recombinant DNA may further comprise a promoter sequence that controls the expression thereof. The choice of promoter was shown to affect the efficiency of recombination in embryonic stem cells transiently transfected with Cre (Araki et al., J. Biochem (Tokyo), 1997, 122: 977-82). Examples of the promoter include, but are not limited to, E. coli lac and trp operons, the tac promoter, the bacteriophage λβ^{P}L promoter, bacteriophage T7 and SP6 promoters, β-actin promoter, insulin promoter, human cytomegalovirus (CMV) promoter, HIV-LTR (HIV-long terminal repeat), Rous sarcoma virus RSV-LTR, simian virus SV40 promoter, baculoviral polyhedrin and p10 promoter. The promoter may also be an inducible promoter that regulates the expression of downstream gene in a controlled manner, such as under a specific condition of the cell culture. Examples of inducible promoters include, but are not limited to, the bacterial dual promoter (activator/repressor expression system) which regulates gene expression in mammalian cells under the control of tetracyclines (Gossen et al., Proc. Natl. Acad. Sci. USA, 89, 5547-5551, 1992) and promoters that regulate gene expression under the control of factors such as heat shocks, steroid hormones, heavy metals, phorbol ester, the adenovirus E1A element, interferon, or serum.

As detailed above, the method of the invention may comprise administering to a patient or to transforming a cell with a composition comprising: a first DNA molecule comprising a first recombination site; and a second DNA molecule comprising a nucleotide sequence encoding at least one recombinase, the at least one recombinase mediates insertion of the nucleotide sequence into a third DNA molecule comprising a second recombination site.

It will be appreciated that various construct schemes can be utilized to deliver the at least one recombinase of the second DNA molecule and further to deliver the first DNA molecules, in a single nucleic acid construct. For example, the two DNA molecules can be co-transcribed as a polycistronic message from a single promoter sequence of the nucleic acid construct. To enable translation of the at least one recombinase from a single polycistronic message, the second DNA molecule can be fused to a linker sequence including an internal ribosome entry site (IRES) sequence, which enables the translation of the polynucleotide segment downstream of the IRES sequence.

According to some embodiments, the site-specific asymmetric recombination is performed by a plurality of recombinases which may comprise at least one distinct wild type recombinase. The wild type recombinase may be derived from prokaryotic and eukaryotic sources.

Site-specific asymmetric recombination according to the present invention may be utilized for genetically modifying plants as plants lack efficient homology recombination. Agrobacterium vector is commonly used for plant transformation, however due to random integration of the transgene the majority of ttansgenic plants confer low transgenic expression and, therefore, non-desired phenotype. Utilization of recombination according to the teaching of the present invention provides an improved methodology resulting in the efficient generation of desirable phenotypes.

It is recognized that many variations of the invention can be practiced. For example, target sites can be constructed having multiple asymmetric recombination sites. Thus, multiple genes or nucleotide sequences can be stacked or ordered at precise locations in the plant genome. Likewise, once a target site has been established within the genome, additional recombination sites may be introduced by incorporating such sites within the nucleotide sequence of the transfer cassette and the transfer of the sites to the target sequence. Thus, once a target site has been established, it is possible to subsequently add sites, or alter sites through recombination.

Another variation includes providing a promoter or transcription initiation region operably linked with the target site in an organism. Preferably, the promoter will be 5′ to the first recombination site. By transforming the organism with a transfer cassette comprising a coding region, expression of the coding region will occur upon integration of the transfer cassette into the target site. This embodiment provides for a method to select transformed cells, particularly plant cells, by providing a selectable marker sequence as the coding sequence.

Therapeutic Applications

Asymmetric recombination of sites using a combination of recombinases may be utilized for treating a variety of diseases. For example, the working of the present invention may be utilized for excision of HIV provirus from the genome of infected cells. Another therapeutic approach includes transforming human bone marrow cells with a combination of recombinases expression constructs. The transformed cells may constitutively or transiently produce the combination of active recombinases thus protecting the cells from HIV infection by an efficient excision of the asymmetric-flanked virus sequence upon infection. Yet, another therapeutic approach is targeting HIV-infected cells by inducing transient expression of the relevant recombinase in the infected cells thereby excising the HIV sequences.

According to yet another aspect, the present invention provides methods of cell therapy using the disclosed genetically modified cells of the invention. Traditional cell therapy approaches utilize ex vivo gene transfer, which involves the initial step of obtaining cells from a subject in need thereof, following transforming of cells in vitro with a desired DNA sequence and finally introducing the transformed cells back into the subject.

Although difficulties in utilizing gene transfer technology and numerous other widely utilized molecular techniques have been reported in the art, successful implementation of these so-called “unpredictable” techniques has been reported in numerous publications, including patent publications that disclose treatment methods utilizing gene therapy.

In many cases, the unpredictability of an art lies in the inability to target the desired gene fragment into a predetermined exact genomic location. The advantage of methods of the present invention is that they provide a targeted recombination and are based on a-priori determination of the insertion or excision genomic locus. Following identification of a desired locus, a recombinase or a plurality of recombinases are selected from a library of recombinases, for example the library disclosed in Santoro et al. (ibid), which can catalyze recombination at the desired locus.

Clearly gene therapy using the methods of the present invention can be applied safely and effectively to a subject in need thereof.

The enzymes of the invention must be internalized in a cell in order to catalyze the desired asymmetric recombination. Cellular internalization of the enzymes of the invention may be achieved by any method known in the art for the delivery of cDNA, peptides and proteins into a cell. Effective delivery of a virus into sites of expression has been demonstrated by numerous studies. For example, internalization may be achieved using an approach utilizing computer-aided tomography (CAT) to direct needle injection into a tumor. This technique has been demonstrated in the treatment of non-small cell lung cancer, by Kauczor et al. ((1999) Eur Radiol 9, 292-296). In a prospective clinical phase I trial, six patients with non-small cell lung cancer and a mutation of the tumor suppressor gene p53 were treated by CAT-guided intratumoral gene therapy. Ten milliliters of a vector solution (replication-defective adenovirus-expressing wild-type p53 cDNA) were injected under CAT guidance. The CAT-guided gene therapy was easily performed in all six patients without intervention-related complications. Besides flu-like symptoms, no significant adverse effects of gene therapy were noted. After 28 days, four of the six patients showed a stable disease at the treated tumor site, whereas other tumor manifestations progressed. This study demonstrated that tomography-guided injection is suitable for performing intratumoral gene therapy.

U.S. Pat. No. 6,652,873 discloses compositions and methods for enhancing receptor-mediated cellular internalization. The compositions include a compound to be delivered and a biocompatible viscous material, such as a hydrogel, lipogel, or highly viscous sol. The composition also include, or are administered in conjunction with, an enhancer in an amount effective to maximize expression of or binding to receptors and enhance receptor-mediated endocytosis (RME) of the compound into the cells. This leads to high transport rates of compounds to be delivered across cell membranes, facilitating more efficient delivery of drugs and diagnostic agents. The enhancer is administered with the composition or separately, either systemically or preferably locally. The compound to be delivered can also be the enhancer.

### Routes of Administration

The compositions of the invention can be administered by any means known in the art, which delivers the composition approximate to the target cells or tissue. These compositions are for use by injection, topical administration, or oral uptake.

Apart from other considerations, the fact that the novel active ingredients of the invention are enzymes or vectors/adenovirus or cells, dictates that the formulation be suitable for delivery of these types of compounds. Clearly, peptides and proteins are less suitable for oral administration due to susceptibility to digestion by gastric acids or intestinal enzymes. It is contemplated that the present invention encompasses compositions designed to circumvent these problems. The preferred routes of administration of enzymes are intra-articular, intravenous, intramuscular, subcutaneous, intradermal, or intrathecal. A more preferred route is by direct injection at or near the site of disorder or disease.

Pharmaceutical compositions of the present invention may be manufactured by processes well known in the art, e.g., by means of conventional mixing, dissolving, granulating, grinding and pulverizing among others.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art. Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The formulations of the enzymes may be administered topically as a gel, ointment, cream, emulsion or sustained release formulation including a transdermal patch. The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art, especially in light of the detailed disclosure provided herein.

Toxicity and therapeutic efficacy of the peptides described herein can be determined by standard pharmaceutical procedures in cell cultures or experimental animals, e.g., by determining the IC₅₀ (the concentration which provides 50% inhibition) and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

### EXAMPLES

### Materials and Methods

### Construction of Wild Type and Mutant LoxP Substrates.

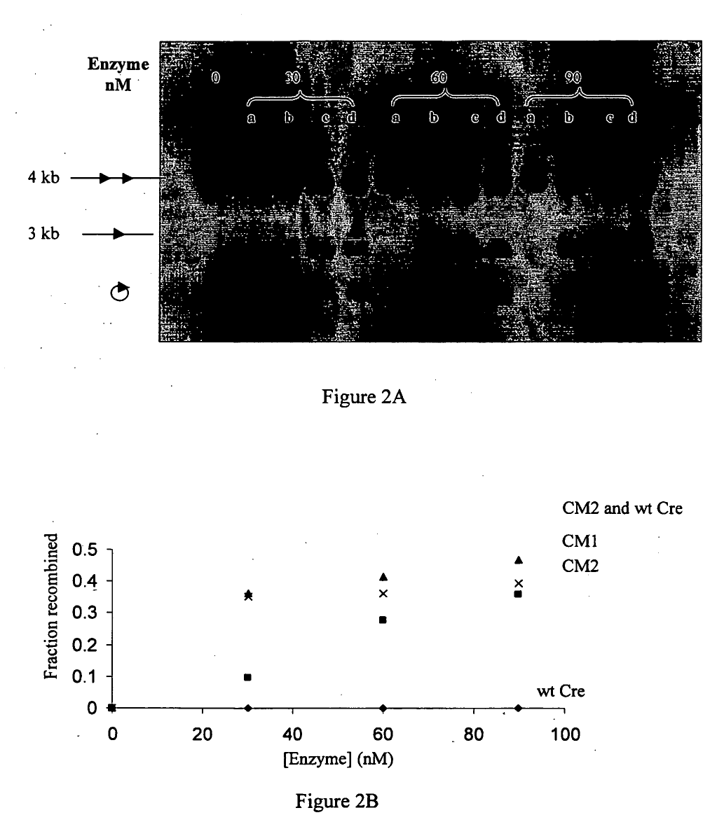

The wild type loxP site and mutant lox M7 site substrate constructs were described previously (Santoro et al., ibid). In this work a chimeric asymmetric lox variant, loxP-M7, was designed to contain one half-site identical to loxP and a second half-site identical to lox M7 (FIG. 1A, spacer sequence in bold and underlined, nucleotide that are different from the wild type site are in bold or underlined). The loxP-M7 substrate plasmid was created by cloning two chimeric loxP-M7 sites (synthesized as oligonucleotides) flanking a 1-kb spacer (BamHI-EcoRI fragment of the nptII gene) into a Bluescript™ vector at the XhoI/PstI restriction sites (FIG. 1C). loxP-M7 sites were cloned in direct orientation (FIG. 2A-B).

### Expression and Purification of Recombinant Cre Variants.

Expression and purification of recombinant wild type and mutant Cre enzymes from bacterial expression vectors harboring wt Cre and the Cre mutants CM1 [C2(+)#1] and CM2 [C2(+/−)#4] was performed as described (Santoro et al., ibid).

### In Vitro Analysis of Recombinase Activity.

In vitro analysis of recombinase activity was performed essentially as described (Santoro et al., ibid). Recombination assays were conducted under conditions of variable reaction time or enzyme concentration. Concentration-dependence assays were performed at enzyme concentrations of 30-90 nM for a fixed reaction time of 1 hr in a reaction buffer containing 300 mM NaCl, 20 mM Tris (pH 7.5) and 1 mM EDTA in a final volume of 40 μL. All reactions were carried out at 37° C. and stopped by incubation at 70° C. for 10 min. Time-course experiments were allowed to proceed for 15-90 min, with 30 nM enzyme concentrations and 1.25 nM of loxP-M7 DNA substrate. Reaction products were analyzed on a 1% agarose gel containing 0.3 μg/μL ethidium bromide and transferred to a Nytransupercharge nylon membrane (Schleicher & Schuell). The 4-kb plasmid containing loxP-M7 substrate linearized with NcoI served as a probe to detect recombination products (FIG. 2A-B).

The 4-kb linear plasmid containing the loxP-M7 chimeric site substrate (50 ng) was random-prime labeled with α-³²P-dATP and products were hybridized to the membrane overnight. Recombination products were visualized by exposure to X-ray film (Kodak) and quantitative results were obtained by processing the membrane using a phosphorimager (FLA-5000, Fuji, Japan) followed by analysis using Image Gauge software V 4.0. Data were fit to the equation: F=F_{eq}(1-e^{-kt}); F=fraction recombined at time t, F_{eq}=maximal fraction recombined at t=∞, and k=the observed rate constant.

### Computational Screen

Human mouse and rat chromosomes Were searched for perfect matches to the 33 spacers (Lee et al., ibid). From each perfect hit, 13 nucleotides upstream and downstream to the spacer were retrieved and analyzed for identity to the loxP sequence. Sequences showing 8 or more nucleotides identical to the loxP sequence both upstream and downstream from the spacer were scored for their palindromic characteristics. The score ranges from 0-13 based upon the number of nucleotides within the 13 nucleotides upstream from the spacer that were reverse complements to the nucleotides within the 13 nucleotides downstream from the spacer. Since the prerequisite for the palindromic score were at least 8 nucleotides identical to the lox sequence on both sides of the spacer, the palindromic score cannot be below 3.

### Example 1

### Design of an In Vitro Recombination Assay for Asymmetric Lox Variant Sites

We approached the question of asymmetric recombination by employing modified Cre recombinase versions recently developed for different lox-related site specificities (Santoro et al., ibid). The experimental design consisted of an in vitro recombination assay which allowed us to monitor the efficiency of recombination obtained using one or a combination of two site-specific Cre-related recombinases on an asymmetric lox site substrate. Specifically, a 4-kb linear double-stranded DNA molecule containing a pair of chimeric asymmetric sites, termed loxP-M7, served as the substrate. Each loxP-M7 site was composed of a wild-type loxP half-site and lox M7 half-site (FIG. 1A). Each member of the pair of lox sites was positioned in direct orientation relative to the other, flanking an intervening ˜1-kb DNA fragment. A recombination event was expected to excise the intervening DNA fragment, resulting in a 3-kb linear fragment and a ˜1-kb circular DNA molecule (FIGS. 2A-B). The activities of recombinant purified wt Cre (specific for loxP), CM1 (equally specific for loxP and lox M7) and CM2 (displaying a 40 fold higher specificity for lox M7 than loxp) enzymes and an equal mixture of wt Cre and CM2 were assayed for the ability to recombine the asymmetric loxP-M7 DNA chimeric substrate. Recombination products were separated by gel electrophoresis and quantified by Southern blot hybridization followed by exposure to film or phosphoimager (FLA-5000, Fuji) and analysis with Image Gauge software V 4.0 (Fuji).

CM1 and CM2 have five substituted amino acids compared with the wt Cre, whereas two of the five are identical in CM1 and CM2 (Table 1).

TABLE 1
Amino acid residues that were modified
in Cre mutants with respect to wt Cre:

| Designation | Residues in wt Cre vs. residues in Cre mutants | | | | |
|---|---|---|---|---|---|
| wt Cre | I174 | T258 | R259 | E262 | E266 |
| CM1 (C2(+)#1) | L174 | N258 | S259 | G262 | G266 |
| CM2 (C2(+/−)#4) | A174 | L258 | S259 | H262 | G266 |

### Example 2

### Recombination of Asymmetric Lox Sites In Vitro

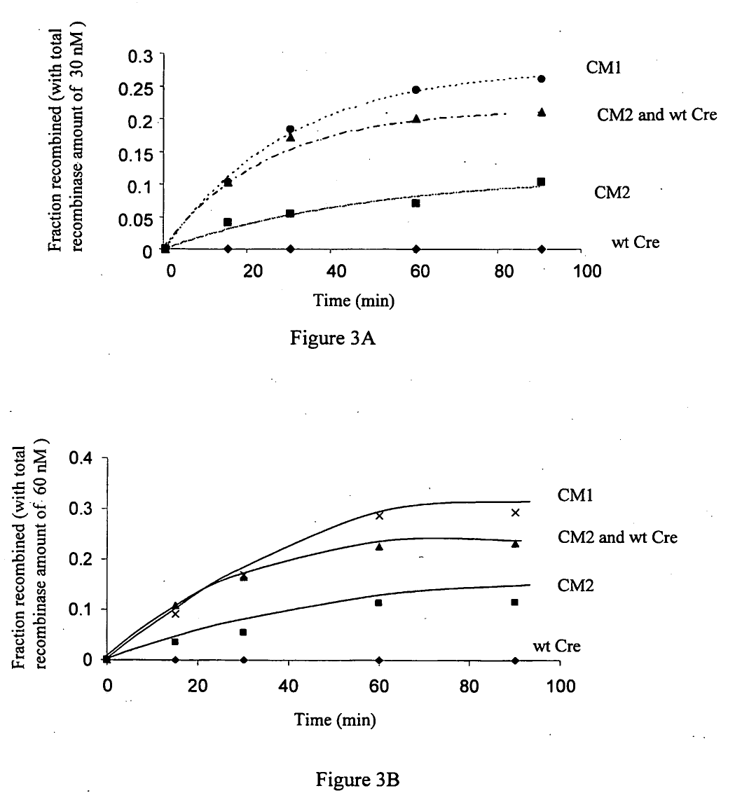

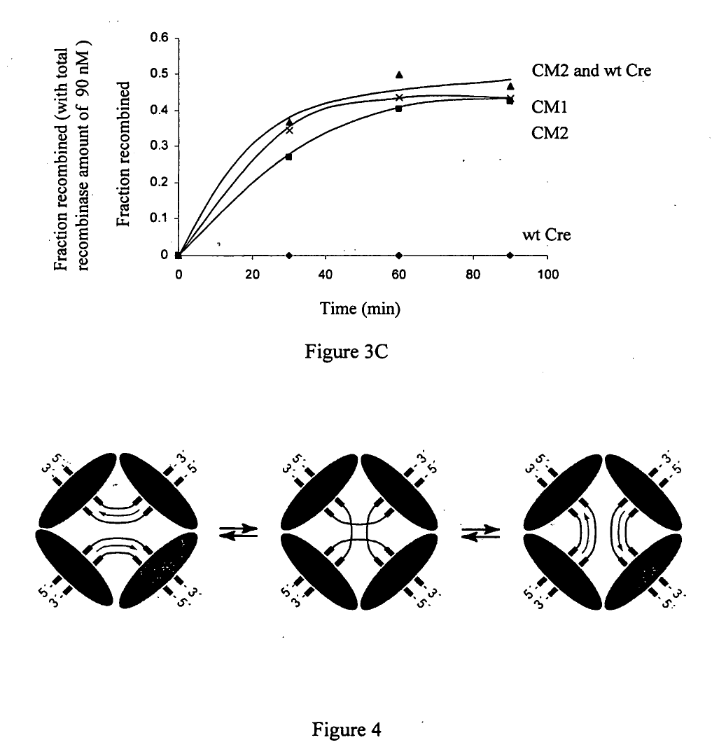

The recombination activities of wt Cre, CM1, CM2 and the wt Cre-CM2 mixture were first assayed at concentrations of 30, 60 and 90 nM with 1.25 nM of the loxP-M7 DNA substrate in a reaction time of 1 h (FIGS. 2A-2B). Wild type Cre exhibited no measurable activity with the loxP-M7 DNA substrate at all enzyme concentrations examined. CM2 exhibited measurable but inefficient activity, recombining 10% of the substrate within the reaction period, when present at a concentration of 30 nM. In contrast, the catalytic efficiencies of CM1 and the wt Cre-CM2 mixture were significantly higher. When present at concentrations of 30 nM and 60 nM, CM1 and the wt Cre-CM2 mixture catalyzed approximately 35% substrate recombination in 1 h (FIGS. 3A-3B).

In order to compare the rates at which wt Cre, CM1, CM2 and the wt Cre-CM2 mixture approach equilibrium in recombining the loxP-M7 asymmetric DNA substrate, a reaction containing 30 nM enzyme and 1.25 nM DNA substrate was allowed to proceed for 90 min. Aliquots were removed and quenched periodically during the reaction period (FIG. 3A). At the 30-nM enzyme concentration, CM2 recombination proceeded at a rate of only 0.020 min-1, reaching 10-15% recombined in 90 min. In contrast, CM1 and the wt Cre-CM2 mixture exhibited significantly higher recombination rates of 0.034 and 0.048 min-1, respectively, and reached recombination extents of approximately 20% after 30 min and 25-30% after 90 min, recombination of the loxP-M7 substrate by wt Cre was not observed. (FIG. 3A).

The experimental results support two major conclusions: (1) A single Cre-related recombinase with relaxed substrate specificity that functions equally efficiently on loxp and lox M7 substrates (CM1) can efficiently catalyze recombination of the chimeric asymmetric substrate. (2) A combination of two different Cre variants (wt and CM2) possessing selective binding specificities for their respective cognate lox half-sites (loxP or lox M7 respectively) can efficiently catalyze recombination of the chimeric loxP-M7 asymmetric substrate.

### Example 3

### The Formation of a Heterotetrameric Structure

The present study demonstrates the feasibility for site-specific recombination of an asymmetric lox site by a combination of two different Cre variants possessing selective binding specificities for their respective cognate half-site on the site. The results presented here strongly suggest that recombination in this system is catalyzed by a heterotetrameric assembly of the two Cre variants (wild type Cre and the CM2 mutant). This conclusion is consistent with the fact that the DNA binding and catalytic domains within Cre reside in two distinct and independent locations on the protein (e.g. Gopaul et al. EMBO J. 1998 17, 4175-4187). This arrangement permits the formation of a recombination synapse involving two asymmetric lox sites aligned in an antiparallel orientation and each bound by a wt Cre-CM2 heterodimer (FIG. 4). The recombination synapse involving two of each Cre variant monomer and two asymmetric lox sites can then go on to form a Holliday junction intermediate, followed by resolution to complete the recombination reaction.

As the results with CM1 illustrate, site-specific recombination of asymmetric target sites can be facilitated by a single Cre-recombinase variant. CM1, a variant of Cre with relaxed substrate specificity that functions equally efficiently with the loxP and lox M7 substrates, also rapidly recombines the asymmetric loxP-M7 substrate when present at a concentration of 30 nM (e.g. FIG. 3A). In contrast, CM2, a recombinase with switched substrate specificity that exhibits ˜40-fold higher recombination efficiency with lox M7 than loxP substrate, reached the catalytic rate of CM1 on the asymmetric substrate only when present at the higher 90 nM enzyme concentration indicating the loss of specificity of CM2 at high concentrations. wt Cre becomes promiscuous in vitro at higher concentration as observed in lox AT (Martin et al. Biochemistry. 2003, 42, 6814-26). Nevertheless, more Cre mutants with higher specificity could be selected for asymmetric lox recombination as heterotetrameric complex. Although recombination of a non-palindromic lox mutant has been previously reported (Rufer et al. Nucleic Acids Res. 2002, 30, 2764-2771) the mutations described were within the “tolerant” region of loxp in nucleotides 11 and 13 (Hartung et al., J. Biol. Chem. 1998, 273, 22884-22891). The present study demonstrate site-specific recombination by a single Cre variant of a non-palindromic site containing mutated nucleotides within the “intolerant” region of loxP in nucleotides 2-7, which are critical for Cre binding.

Interestingly, both the wt Cre-CM2 mixture and CM1 demonstrated comparable catalytic efficiency, suggesting that the heterotetrameric assembly of Cre variants imposes no penalty on recombination efficiency. Because Cre variants with relaxed specificity such as CM1 could in principle recognize non-targeted lox-related sites, such variants could result in off-target effects. In contrast, the use of two different highly-specific Cre variants permits the targeting of asymmetric sites with minimal risk of off-target activities.

### Example 4

### Applications of the Heterotetrameric Formation and Recombination on Natural Asymmetric Sites

In order to study the potential applications of asymmetric recombination in vivo, we employed a bioinformatics approach to detect potential lox-variant sites within mammalian genomes. Wt Cre has been shown to tolerate changes within the 8 bp spacer region (Table 2; Lee et al., ibid).

TABLE 2

Mutants of the lox spacer (Lee et al., Gene.
ibid) and wild-type lox spacer

| | |
|---|---|
| | SEQ ID |
| Sequence | NO |

| | |
|---|---|
| ACGTATGC | 1 |
| AAGTATGC | 2 |
| AGGTATGC | 3 |
| ATATATGC | 4 |
| ATCTATGC | 5 |
| ATTTATGC | 6 |
| ATGCATGC | 7 |
| ATGAATGC | 8 |
| ATGGATGC | 9 |
| ATGTGTGC | 10 |
| ATGTTTGC | 11 |
| ATGTCTGC | 12 |
| ATGTACGC | 13 |
| ATGTAGGC | 14 |
| ATGTATAC | 15 |
| ACGTATGC | 16 |
| AAGTATGC | 17 |
| AGGTATGC | 18 |
| ATGTATAC | 19 |
| ATGTATCC | 20 |
| ATGTATTC | 21 |
| ACGTATAC | 22 |
| ATATATAC | 23 |
| ATGCATAC | 24 |
| ATGTGTAC | 25 |
| AAGTATCC | 26 |
| ATCTATCC | 27 |
| ATGAATCT | 28 |
| ATGTTTCC | 29 |
| AGTTATTC | 30 |
| ATTTATAC | 31 |
| ATGGATTC | 32 |
| ATGTCTTC | 33 |
| ATGTATGC | 34 |

Computational screen in rat, mouse and human for potential lox like sites containing a 33 tolerated spacers, revealed several hundred asymmetric lox like sites versus only a couple of symmetric lox like sites within the genome of rat, mouse and human. (Table 3). Numbers are corresponding to bases identity of loxP in left or right side of the 13 inverted repeats. Bold numbers represent symmetric sites. Right and left corresponds to loxP right and left arbitrary sides to the spacer.

TABLE 3
Computational screen for asymmetric
vs. symmetric lox like genomic sites
Right side

| | | 8 | 9 | 10 | 11 | 12 | 13 |
|---|---|---|---|---|---|---|---|
| | Human | | | | | | |
| Left Side | 8 | 243/1 | 46 | 4 | 2 | 0 | 0 |
| | 9 | 37 | 11 | 1 | 0 | 0 | 0 |
| | 10 | 3 | 0 | 0 | 0 | 0 | 0 |
| | 11 | 0 | 0 | 0 | 0 | 0 | 0 |
| | 12 | 0 | 0 | 0 | 0 | 0 | 0 |
| | 13 | 0 | 0 | 0 | 0 | 0 | 0 |
| | Mouse | | | | | | |
| | 8 | 181 | 36 | 3 | 1 | 0 | 0 |
| | 9 | 39 | 2 | 2 | 0 | 0 | 0 |
| | 10 | 5 | 3 | 0 | 0 | 0 | 0 |
| | 11 | 0 | 0 | 0 | 0 | 0 | 0 |
| | 12 | 0 | 0 | 0 | 0 | 0 | 0 |
| | 13 | 0 | 0 | 0 | 0 | 0 | 2 |
| | Rat | | | | | | |
| | 8 | 145 | 31 | 6 | 1 | 0 | 0 |
| | 9 | 28 | 5 | 0 | 0 | 0 | 0 |
| | 10 | 6 | 1 | 0 | 0 | 0 | 0 |
| | 11 | 0 | 0 | 0 | 0 | 0 | 0 |
| | 12 | 0 | 0 | 0 | 0 | 0 | 0 |
| | 13 | 0 | 0 | 0 | 0 | 0 | 0 |
| | | 0 | 0 | 0 | 0 | 0 | 2 |

### Example 5

### Selection of New Cre Variants that Facilitate Integration of Exogenous DNA into Endogenous Lox-Like Site

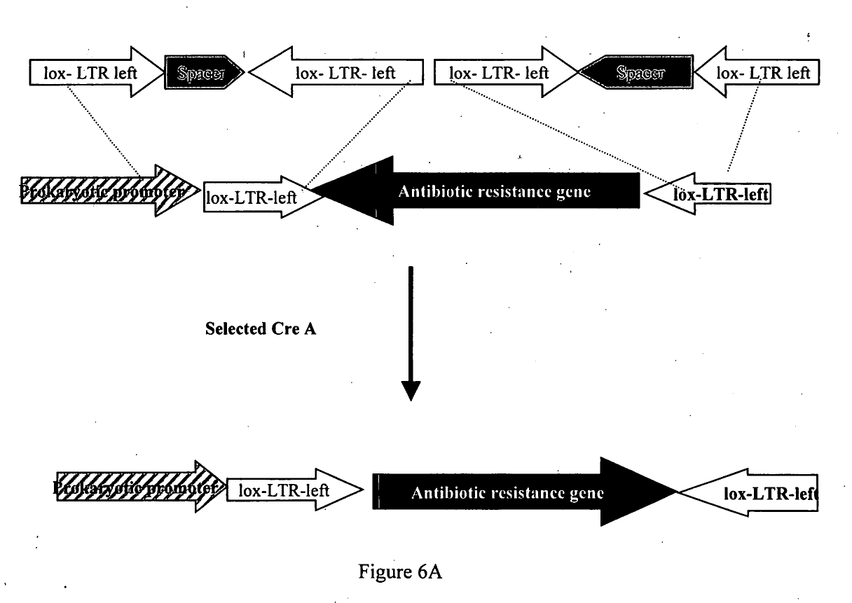

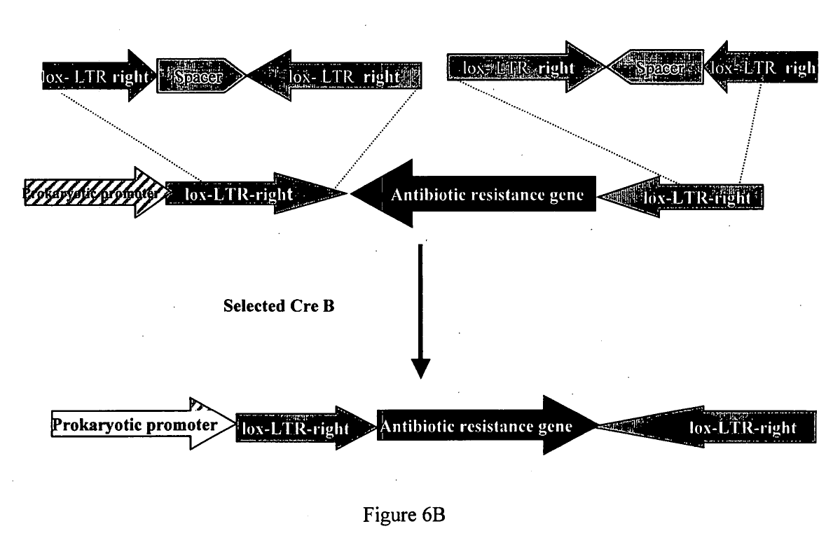

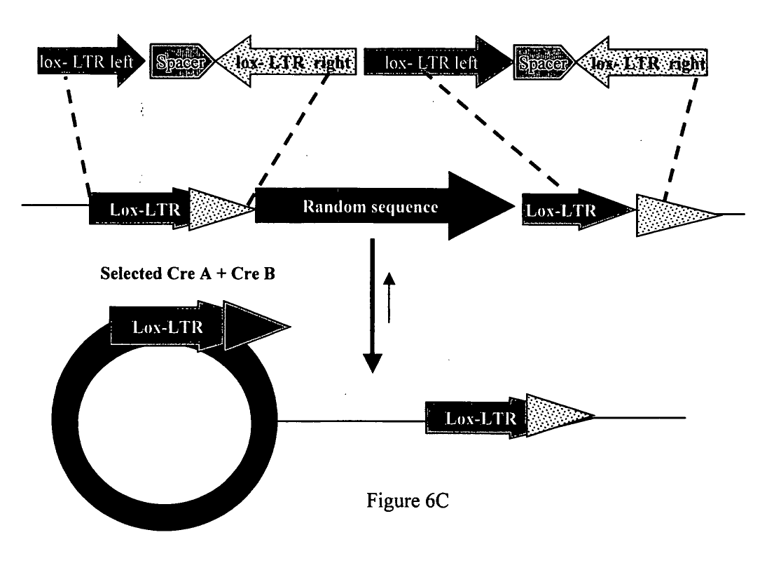

Selection of suitable cells expressing the desired recombinase is attributed to the orientation of the promoter. Since the antibiotic resistance gene is orientated ‘wrongly’, i.e. in an opposite orientation to the promoter, it is not transcribed unless inversion recombination is facilitated by a competent Cre mutant (FIG. 6). Thus, a desired Cre mutant gives rise to transcription of the antibiotic resistance gene thereby conferring antibiotic resistance to the cell comprising thereof. The desired Cre mutants are then tested in-vitro and in-vivo, individually or as a combination of a plurality of desired Cre mutants. It is suggested that asymmetric recombination is facilitated by a plurality of desired Cre mutants since a heterotetrameric structure of the plurality of desired Cre mutants is formed.

### Example 6

### Selection Strategy for Cre Mutants that Facilitate Genetic Antiviral Therapy Against HIV

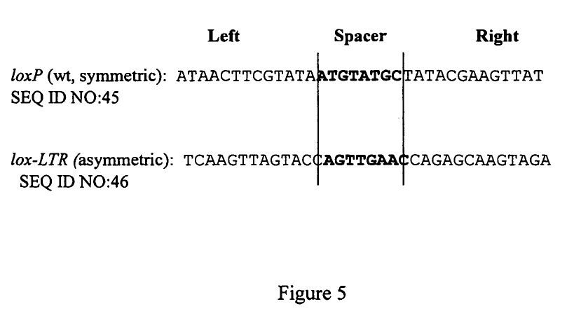

In retroviruses, LTR sequences flank the virus sequence from both sides. Lee et al. (Lee Y, et al., Biochem. Biophys. Res. Commun. 253:588-93, 1998) found a sequence within the LTR which has some homology to loxP, this sequence is also termed “lox-LTR” (FIG. 5; SEQ ID NO:46). The spacer region of the lox-LTR was cloned into a loxP replacing the wt spacer and the new substrate was recognized by wt Cre to facilitate recombination.

Since lox-LTR sites are asymmetric, the selection strategy for Cre mutants that can recognize and catalyze specific recombination in these lox-LTR sites began with the design of two lox-LTR derivatives as follows: In the first derivative, the half left site was placed on both sides of the spacer, in opposite directions (FIG. 6A) and in the second derivative, the right half of the site was put on both sides of the spacer, in opposite directions (FIG. 6B). Each of these two symmetric lox-LTR derivatives was cloned within a construct in opposite directions on both sides of an antibiotic selectable gene (see FIGS. 6A-5B). In this construct, the lox-LTR site was inserted between a bacterial promoter and an antibiotic resistance gene, which was positioned in opposite direction to the promoter (FIGS. 6A and 6B).

For the selection of new Cre mutants, the two constructs described in FIGS. 6A-5B are transformed into a Cre mutants library cells (Santoro et al., ibid). The antibiotic resistance gene is positioned in an opposite orientation to the promoter, and therefore is not transcribed. However, in the presence of competent Cre mutants, inversion recombination is facilitated (FIG. 6B), giving rise to transcription and conferring antibiotic resistance to the bacteria. Positive Cre mutants that mediated recombination of constructs as described in FIGS. 5A and 5B are tested, individually and as a combination, in-vitro and in-vivo. The combination of a plurality of Cre mutants forms a heterotetrameric structure thereby facilitating recombination of a natural, asymmetric lox-LTR (FIG. 6C). A preferred strategy is directed to the selection of Cre mutants that facilitate genetic antiviral therapy against HIV by excising the viral genome flanked between viral LTR recombination sites.

### Example 7

### Selection Strategy for LTR Assay

### Construction of LTR Reporter Plasmids:

Plasmid PS-LoxP (Santoro et al., ibid) was used, with the following modifications:
1. The GFPuv gene was replaced by chloramphenicol resistance gene in the opposite direction. Cre mutant's library was transformed into cells containing PS plasmid. Cre mutant was selected as a result of recombination event and antibiotic resistance.
2. Primers containing the LTR's attached to a 20 bases sequence from the chloramphenicol or the EYFP genes (Enhanced Expression of Yellow Fluorescent Protein) were designed. The PCR products produced using these primers and the PS-Chloramphenicol. plasmid as a template were re-ligated to the plasmid at EcoRI/XbaI sites, allowing the LTR sequences to replace the LoxP sites. The following substrates for the LTR assay were used (in bold are nucleotide that do not exist in the wild type LoxP):
   LoxP (SEQ ID NO:35): ATAACTTCGTATAGCATACATTATACGAAGTTAT
   LTR1 (SEQ ID NO:47): TCAAGTTAGTACCGTTCAACTGGTACTAACTTGA
   LTR2 (SEQ ID NO:48): TCTACTTGCTCTGGTTCAACTCAGAGCAAGTAGA
   Substrates with 3-4 mutations:
   LTR1-1 (SEQ ID NO:49): ATAACTTAGTACCGCATACATGGTACTAAGTTAT
   LTR1-2 (SEQ ID NO:50): TCAAGTTCGTATAGCATACATTATACGAACTTGA
   LTR2-1 (SEQ ID NO:51): ATAACTTGCTCTGGCATACATCAGAGCAAGTTAT
   LTR2-2 (SEQ ID NO:52): TCTACTTCGTATAGCATACATTATACGAAGTAGA
   Substrates with 2-3 mutations (the numbers indicates the locations of the mutations):
   LTR1-1113 (SEQ ID NO:53): TCAACTTCGTATAGCATACATTATACGAAGTTGA
   LTR1-69 (SEQ ID NO:54): ATAAGTTAGTATAGCATACATTATACTAACTTAT
   LTR1-12 (SEQ ID NO:55): ATAACTTCGTACCGCATACATGGTACGAAGTTAT
   LTR2-56 (SEQ ID NO:56): ATAACTTGCTATAGCATACATTATAGCAAGTTAT
   LTR2-13 (SEQ ID NO:57): ATAACTTCGTCTGGCATACATCAGACGAAGTTAT

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-71. (canceled)

72. An isolated enzyme capable of mediating a site-specific recombination between two predetermined recombination sites, wherein at least one recombination site is an asymmetric recombination site.

73. The isolated enzyme according to claim 72, wherein the recombination is selected from a group consisting of: inversion of a first DNA molecule encompassed within a second DNA molecule, excision of a first DNA molecule from a second DNA molecule, insertion of a first DNA molecule into a second DNA molecule and translocation between a first DNA molecule and a second DNA molecule, wherein the second DNA molecule is selected from the group consisting of: genomic DNA and circular DNA.

74. The isolated enzyme according to claim 73, wherein the second DNA molecule is genomic DNA and the first DNA molecule is integrated into a predetermined genomic site selected from the group consisting of: 3′ UTRs, 5′ UTRs, polyA sites and gene promoters.

75. The isolated enzyme according to claim 72, wherein said isolated enzyme is a Cre or FLP mutant mediating recombination between two recombination sites, such that at least one recombination site is an asymmetric recombination site comprising a spacer sequence selected from the group consisting of: SEQ ID NOS. 1-34.

76. A plurality of isolated enzymes capable of mediating site-specific recombination between two predetermined recombination sites, wherein at least one of the recombination sites is an asymmetric recombination site, wherein at least one enzyme is selected from the group consisting of: a wild type recombinase, Cre and Flp mutant, and at least one recombination site is an asymmetric recombination site comprising a spacer sequence selected from the group consisting of: SEQ ID NOS. 1-34.

77. An isolated polynucleotide encoding the at least one enzyme of claim 76, wherein said isolated polynucleotide is encompassed in a recombinant vector that expresses the at least one recombinase and is selected from the group consisting of: naked DNA plasmid, a plasmid within a liposome, a retroviral vector, an AAV vector, or a recombinant adenoviral vector.

78. The isolated polynucleotide according to claim 77, wherein the recombinant vector further comprising a promoter derived from bacteria, yeast, insect, animal, plant and virus and selected from the group consisting of: E. coli lac and trp operons, the tac promoter, the bacteriophage _{λ}L promoter, bacteriophage T7 and SP6 promoters, β-actin promoter, insulin promoter, human cytomegalovirus (CMV) promoter, HIV-LTR, RSV-LTR, SV40 promoter, baculoviral polyhedrin and p10 promoter.

79. The isolated polynucleotide according to claim 78, wherein the promoter is an inducible promoter selected from the group consisting of: tetracycline, heat shock, steroid hormone, heavy metal, phorbol ester, adenovirus E1A element, interferon and serum inducible promoters.

80. The isolated polynucleotide according to claim 77, wherein said isolated polynucleotide encodes a plurality of enzymes, the plurality of enzymes is capable of mediating site-specific recombination between two predetermined recombination sites, wherein at least one of the recombination sites is an asymmetric recombination site and each of the plurality of recombinases recognizes at least one half of the at least one asymmetric recombination site.

81. A host cell comprising the polynucleotide of claim 77.

82. A genetically modified cell transformed by a site-specific recombination between two recombination sites carried out by the isolated enzyme of claim 72, wherein at least one of the recombination sites is an asymmetric recombination site, and wherein the asymmetric recombination is selected from the group consisting of: inversion, excision, insertion and translocation, wherein the recombination occurs between the cellular endogenous genome and an exogenous DNA molecule such that the exogenous DNA molecule is integrated by recombination between the two recombination sites into a predetermined locus within the cellular genome.

83. The genetically modified cell according to claim 82, wherein said genetically modified cell is eukaryotic, wherein said genetically modified cell is selected from the group consisting of: yeast, plant cell, embryonic stem cell, mesenchymal cell, and haematopoietic progenitor cell.

84. A transgenic organism comprising the genetically modified cell of claim 83, said transgenic organism is selected from the group consisting of: plant, yeast and mammal.

85. The genetically modified cell according to claim 83, wherein said cell is devoid of an endogenous polynucleotide sequence at a predetermined genomic locus.

86. A method for treating a disease, comprising:
a. providing a composition comprising a DNA molecule comprising a nucleotide sequence encoding at least one enzyme, the at least one enzyme is capable of mediating site-specific excision of a gene fragment flanked between two recombination sites, wherein at least one recombination site is an asymmetric recombination site;
b. administering the composition to a subject in need thereof; thereby obtaining site-specific excision of the gene fragment from a predetermined genomic locus.
wherein the composition further comprises a carrier operably connected to the isolated DNA molecule, the carrier capable of targeting said isolated DNA molecule to a cell and promoting internalization of said isolated DNA molecule into the cell and said carrier is selected from the group consisting of: viruses, liposomes, lipid/DNA complexes, micelles, protein/lipid complexes, nanoparticles, and microparticles.

87. The method according to claim 86, wherein the two recombination sites are the same asymmetric recombination sites.

88. The method according to claim 86, wherein the nucleotide sequence encodes a plurality of enzymes capable of catalyzing the recombination.

89. The method according to claim 86, wherein the excised gene fragment is a fragment of HIV genomic DNA.

90. The method according to claim 86, wherein the composition comprises a recombinant vector encompassing an expression cassette comprising the nucleotide sequence, wherein the vector is selected from the group consisting of: naked DNA plasmid, a plasmid within a liposome, retrovirus, lentivirus, adenovirus, herpes simplex viruses (HSV), cytomegalovirus (CMV), and adeno-associated virus (AAV).

91. The method of claim 86, comprising:
a. providing a composition comprising a DNA molecule comprising a nucleotide sequence encoding at least one enzyme, the at least one enzyme is capable of mediating site-specific excision of a gene fragment flanked between two recombination sites at a defined genomic locus, wherein at least one recombination site is an asymmetric recombination site;
b. transforming a cell with the composition; and
c. proliferating the transformed cells ex vivo.
wherein the cell is autologous.

92. The method according to claim 91, further comprising: selecting cells devoid of said gene fragment and transplanting the selected cell into a subject in need thereof.

93. The method according to claim 91, wherein transforming the cell with said composition is carried out by a procedure selected from the group consisting of: calcium phosphate transfection, DEAE-dextran mediated transfection, transvection, microinjection, cationic lipid-mediated transfection, electroporation, scrape loading, ballistic introduction or infection, use of a gene gun and liposome transfection.

94. The method of claim 86 wherein the composition comprises a first DNA molecule comprising a first recombination site; and a second DNA molecule comprising a nucleotide sequence encoding at least one enzyme, the at least one enzyme mediates insertion of the first DNA molecule or fragments thereof into a third DNA molecule comprising a second recombination site, wherein the third DNA molecule is genomic DNA such that the first DNA molecule is inserted into a defined locus of the genome selected from the group consisting of: 3′ UTRs, 5′ UTRs, polyA sites and gene promoters.

95. The method according to claim 94, wherein the first DNA molecule comprises a nucleotide sequence consisting of a fragment of human genomic DNA encoding for a molecule selected from the group consisting of: a structural protein, an enzyme and a regulatory molecule.

96. The method according to claim 94, wherein the composition further comprises a carrier operably connected to the first and second DNA molecules, the carrier capable of targeting said first and second DNA molecules to a cell encompassing the third DNA molecule, wherein the carrier is selected from the group consisting of: viruses, liposomes, lipid/DNA complexes, micelles, protein/lipid complexes, nanoparticles and microparticles.

97. The method according to claim 94, wherein the first DNA molecule and the second DNA molecule are operatively linked to one another and the second DNA molecule is operably linked to a promoter.

98. The method according to claim 94, wherein the first DNA molecule comprises a recombination site comprising SEQ ID NO:37 and the second DNA molecule comprising a nucleotide sequence encoding at least one enzyme selected from the group consisting of: wild type Cre, CM1 Cre mutant and CM2 Cre mutant.

99. The method according to claim 94, further comprising selecting cells comprising the first DNA molecule integrated within their genome at a predetermined locus and transplanting the cell into a subject in need thereof.

100. The method according to claim 86, wherein the composition comprises:
(i) a first DNA molecule, the first DNA molecule comprises a first recombination site; and
(ii) at least one enzyme capable of mediating site-specific insertion of the first DNA molecule into a second recombination site within a specific genomic locus, wherein at least one of said first and second recombination site is an asymmetric recombination site
thereby obtaining at step (b) a site-specific excision of the gene fragment from a predetermined genomic locus.

101. The method according to claim 100, wherein the first DNA molecule comprises a recombination site comprising SEQ ID NO:37 and the at least one enzyme is selected from the group consisting of: wild type Cre, CM1 Cre mutant and CM2 Cre mutant.


SEQUENCE LISTING (55 sequences)

SEQ ID NO: 1 - DNA, 8 bp, Artificial
  mutant loxP spacer
acgtatgc

SEQ ID NO: 2 - DNA, 8 bp, Artificial
  Mutant LoxP spacer
aagtatgc

SEQ ID NO: 3 - DNA, 8 bp, Artificial
  Mutant LoxP spacer
aggtatgc

SEQ ID NO: 4 - DNA, 8 bp, Artificial
  Mutant LoxP site
atatatgc

SEQ ID NO: 5 - DNA, 8 bp, Artificial
  Mutant LoxP spacer
atctatgc

SEQ ID NO: 6 - DNA, 8 bp, Artificial
  Mutant LoxP spacer
atttatgc

SEQ ID NO: 7 - DNA, 8 bp, Artificial
  Mutant Lox P spacer
atgcatgc

SEQ ID NO: 8 - DNA, 8 bp, Artificial
  Mutant LoxP spacer
atgaatgc

SEQ ID NO: 9 - DNA, 8 bp, Artificial
  Mutant LoxP spacer
atggatgc

SEQ ID NO: 10 - DNA, 8 bp, Artificial
  Mutant LoxP spacer
atgtgtgc

SEQ ID NO: 11 - DNA, 8 bp, Artificial
  Mutant LoxP spacer
atgtttgc

SEQ ID NO: 12 - DNA, 8 bp, Artificial
  Mutant LoxP spacer
atgtctgc

SEQ ID NO: 13 - DNA, 8 bp, Artificial
  Mutant LoxP spacer
atgtacgc

SEQ ID NO: 14 - DNA, 8 bp, Artificial
  Mutant LoxP spacer
atgtaggc

SEQ ID NO: 15 - DNA, 8 bp, Artificial
  Mutant LoxP spacer
atgtatac

SEQ ID NO: 16 - DNA, 8 bp, Artificial
  Mutant LoxP spacer
acgtatgc

SEQ ID NO: 17 - DNA, 8 bp, Artificial
  Mutant LoxP spacer
aagtatgc

SEQ ID NO: 18 - DNA, 8 bp, Artificial
  Mutant LoxP spacer
aggtatgc

SEQ ID NO: 19 - DNA, 8 bp, Artificial
  Mutant LoxP spacer
atgtatac

SEQ ID NO: 20 - DNA, 8 bp, Artificial
  Mutant LoxP spacer
atgtatcc

SEQ ID NO: 21 - DNA, 8 bp, Artificial
  Mutant LoxP spacer
atgtattc

SEQ ID NO: 22 - DNA, 8 bp, Artificial
  Mutant LoxP spacer
acgtatac

SEQ ID NO: 23 - DNA, 8 bp, Artificial
  Mutant LoxP spacer
atatatac

SEQ ID NO: 24 - DNA, 8 bp, Artificial
  Mutant LoxP spacer
atgcatac

SEQ ID NO: 25 - DNA, 8 bp, Artificial
  Mutant LoxP spacer
atgtgtac

SEQ ID NO: 26 - DNA, 8 bp, Artificial
  Mutant LoxP spacer
aagtatcc

SEQ ID NO: 27 - DNA, 8 bp, Artificial
  Mutant LoxP spacer
atctatcc

SEQ ID NO: 28 - DNA, 8 bp, Artificial
  Mutant LoxP spacer
atgaatct

SEQ ID NO: 29 - DNA, 8 bp, Artificial
  Mutant LoxP spacer
atgtttcc

SEQ ID NO: 30 - DNA, 8 bp, Artificial
  Mutant LoxP spacer
agttattc

SEQ ID NO: 31 - DNA, 8 bp, Artificial
  Mutant LoxP spacer
atttatac

SEQ ID NO: 32 - DNA, 8 bp, Artificial
  Mutant LoxP spacer
atggattc

SEQ ID NO: 33 - DNA, 8 bp, Artificial
  Mutant LoxP spacer
atgtcttc

SEQ ID NO: 34 - DNA, 8 bp, Artificial
  Mutant LoxP spacer
atgtatgc

SEQ ID NO: 35 - skipped

SEQ ID NO: 36 - DNA, 46 bp, Bacteriophage P1
  misc_feature (1)..(6): XhoI - restriction site
  misc_feature (41)..(46): BamH1 - restriction site
ctcgagataacttcgtatagcatacattatacgaagttatggattc

SEQ ID NO: 37 - DNA, 46 bp, Bacteriophage P1
  misc_feature (1)..(6): EcoR1-restriction site
  misc_feature (41)..(46): Pst1-restriction site
gaattcataacttcgtatagcatacattatacgaagttatctgcag

SEQ ID NO: 38 - DNA, 46 bp, Artificial
  a symmetric recombination site containing mutant loxM7 palindromic repeats
  misc_feature (1)..(6): Xho1-restriction site
  misc_feature (41)..(46): BamH1-restriction site
ctcgagataactctatatagcatacattatatagagttatggattc

SEQ ID NO: 39 - DNA, 46 bp, Artificial
  a symmetric recombination site containing mutant loxM7 palindromic repeats
  misc_feature (1)..(6): EcoR1-restriction site
  misc_feature (41)..(46): Pst1-restriction site
gaattcataactctatatagcatacattatatagagttatctgcag

SEQ ID NO: 40 - DNA, 46 bp, Artificial
  an assymmetric recombination site containing one half of mutant loxM7 and another half of WT LoxP (non-palindromic repeats)
  misc_feature (1)..(6): Xho1-restriction site
  misc_feature (41)..(46): BamH1-restriction site
ctcgagataacttcgtatagcatacattatatagagttatggattc

SEQ ID NO: 41 - DNA, 46 bp, Artificial
  an assymmetric recombination site containing one half of mutant loxM7 and another half of WT LoxP (non-palindromic repeats)
  misc_feature (1)..(6): EcoR1-restriction site
  misc_feature (41)..(46): Pst1-restriction site
gaattcataacttcgtatagcatacattatatagagttatctgcag

SEQ ID NO: 42 - DNA, 34 bp, Artificial
  loxM7 (Santoro et al., 2002,  Proc. Natl. Acad. Sci. USA, 99: 4185-4190, 2002).
ataactctatatagcatacattatatagagttat

SEQ ID NO: 43 - DNA, 34 bp, Artificial
  an asymmetric recombination site consiting of one half of loxP WT and one half of loxM7 (Santoro et al., ibid)
ataacttcgtatagcatacattatatagagttat

SEQ ID NO: 44 - DNA, 34 bp, Artificial
  an asymmetric recombination site consiting of one half of loxP WT and one half of loxM7 (Santoro et al., ibid)
ataactctatatagcatacattatacgaagttat

SEQ ID NO: 45 - DNA, 34 bp, Artificial
  mutant LTR site (LTR1)
tcaagttagtaccgttcaactggtactaacttga

SEQ ID NO: 46 - DNA, 34 bp, Artificial
  mutant LTR recombination site (LTR2)
tctacttgctctggttcaactcagagcaagtaga

SEQ ID NO: 47 - DNA, 34 bp, Artificial
  mutant LTR recombination site
ataacttagtaccgcatacatggtactaagttat

SEQ ID NO: 48 - DNA, 34 bp, Artificial
  mutant LTR recombination site
tcaagttcgtatagcatacattatacgaacttga

SEQ ID NO: 49 - DNA, 34 bp, Artificial
  mutant LTR recombination site
ataacttgctctggcatacatcagagcaagttat

SEQ ID NO: 50 - DNA, 34 bp, Artificial
  mutant LTR recombination site
tctacttcgtatagcatacattatacgaagtaga

SEQ ID NO: 51 - DNA, 34 bp, Artificial
  mutant LTR recombination site
tcaacttcgtatagcatacattatacgaagttga

SEQ ID NO: 52 - DNA, 34 bp, Artificial
  mutant LTR recombination site
ataagttagtatagcatacattatactaacttat

SEQ ID NO: 53 - DNA, 34 bp, Artificial
  mutant LTR recombination site
ataacttcgtaccgcatacatggtacgaagttat

SEQ ID NO: 54 - DNA, 34 bp, Artificial
  mutant LTR recombination site
ataacttgctatagcatacattatagcaagttat

SEQ ID NO: 55 - DNA, 34 bp, Artificial
  mutant LTR recombination site
ataacttcgtctggcatacatcagacgaagttat